SEQUENCE LISTING (118 sequences)

SEQ ID NO: 1 - protein, 113 aa, synthetic construct
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEAGNDMRFGAGTRLTVKP

SEQ ID NO: 2 - protein, 112 aa, synthetic construct
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSYLWSYEQYFGPGTRLTVT

SEQ ID NO: 3 - protein, 113 aa, synthetic construct
AQKVTQSQTELSVVEGEDVTIDCVYETRDTTYYLFWYKQPPSGEPVFLIRRNSFDEQNEISGRYSWNFQKSTSSFNFTITDVQPNDSAVYFCALSEAGNDMRFGAGTRLTVKP

SEQ ID NO: 4 - protein, 112 aa, synthetic construct
NAGVTQTPKYLSVKTGQSVTLQCAQDMNHEYMYWYRQDPGQGLRLIYYSVGEGTTAKGEVPDRYNVSRLKKQNFLLGIESVTPSDTSVYFCASSYLWSYEQYFGPGTRLTVT

SEQ ID NO: 5 - DNA, 339 bp, synthetic construct
  source 1..339: mol_type = other DNA
gctcaaaaagttactcaaagccaaaccgagctgagcgtggttgagggtgaagacgtgaccatcgattgcgtttatgaaacccgtgacaccacctactacctgttctggtacaagcaaccgccgagcggcgagccggttttcctgatccgtcgtaacagctttgatgagcagaacgaaattagcggccgttatagctggaactttcagaagagcaccagcagcttcaactttaccattaccgacgtgcagccgaacgatagcgcggtttacttctgcgcgctgagcgaagcgggtaacgacatgcgttttggtgcgggtacccgtctgaccgtgaaaccg

SEQ ID NO: 6 - DNA, 336 bp, synthetic construct
  source 1..336: mol_type = other DNA
aacgcgggcgttacccagaccccgaagtatctgagcgtgaaaaccggtcaaagcgttaccctgcagtgcgcgcaagacatgaaccacgagtacatgtattggtaccgtcaggacccgggtcaaggcctgcgtctgatctactatagcgtgggcgagggcaccaccgcgaaaggtgaagtgccggaccgttacaacgttagccgtctgaagaaacagaacttcctgctgggcattgagagcgtgaccccgagcgataccagcgtttatttctgcgcgagcagctacctgtggagctatgaacaatactttggtccgggcacccgtctgaccgttacc

SEQ ID NO: 7 - protein, 24 aa, synthetic construct
GGGSEGGGSEGGGSEGGGSEGGTG

SEQ ID NO: 8 - DNA, 72 bp, synthetic construct
  source 1..72: mol_type = other DNA
ggtggcggtagcgagggcggtggcagcgaaggtggcggtagcgagggcggtggcagcgaaggtggcaccggt

SEQ ID NO: 9 - protein, 249 aa, synthetic construct
AQKVTQSQTELSVVEGEDVTIDCVYETRDTTYYLFWYKQPPSGEPVFLIRRNSFDEQNEISGRYSWNFQKSTSSFNFTITDVQPNDSAVYFCALSEAGNDMRFGAGTRLTVKPGGGSEGGGSEGGGSEGGGSEGGTGNAGVTQTPKYLSVKTGQSVTLQCAQDMNHEYMYWYRQDPGQGLRLIYYSVGEGTTAKGEVPDRYNVSRLKKQNFLLGIESVTPSDTSVYFCASSYLWSYEQYFGPGTRLTVT

SEQ ID NO: 10 - DNA, 747 bp, synthetic construct
  source 1..747: mol_type = other DNA
gctcaaaaagttactcaaagccaaaccgagctgagcgtggttgagggtgaagacgtgaccatcgattgcgtttatgaaacccgtgacaccacctactacctgttctggtacaagcaaccgccgagcggcgagccggttttcctgatccgtcgtaacagctttgatgagcagaacgaaattagcggccgttatagctggaactttcagaagagcaccagcagcttcaactttaccattaccgacgtgcagccgaacgatagcgcggtttacttctgcgcgctgagcgaagcgggtaacgacatgcgttttggtgcgggtacccgtctgaccgtgaaaccgggtggcggtagcgagggcggtggcagcgaaggtggcggtagcgagggcggtggcagcgaaggtggcaccggtaacgcgggcgttacccagaccccgaagtatctgagcgtgaaaaccggtcaaagcgttaccctgcagtgcgcgcaagacatgaaccacgagtacatgtattggtaccgtcaggacccgggtcaaggcctgcgtctgatctactatagcgtgggcgagggcaccaccgcgaaaggtgaagtgccggaccgttacaacgttagccgtctgaagaaacagaacttcctgctgggcattgagagcgtgaccccgagcgataccagcgtttatttctgcgcgagcagctacctgtggagctatgaacaatactttggtccgggcacccgtctgaccgttacc

SEQ ID NO: 11 - protein, 207 aa, synthetic construct
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEAGNDMRFGAGTRLTVKPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 12 - protein, 242 aa, synthetic construct
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSYLWSYEQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 13 - protein, 113 aa, synthetic construct
AQKVTQAQTEISVVEKEDVTLDCVYETRDTDYYLFWYKQPPSGELVFLIRRNSFDEQNEISGRYSWNFQKSTSSFNFTITASQVVDSAVYFCALSEAGNDMRFGAGTRLTVKP

SEQ ID NO: 14 - protein, 113 aa, synthetic construct
AQKVTQAQTEISVVEKEDVTLDCVYETRDTNYYLFWYKQPPSGELVFLIRRNSFDEQNEISGRYSWNFQKSTSSFNFTITASQVVDSAVYFCALSEAGNDMRFGAGTRLTVKP

SEQ ID NO: 15 - protein, 113 aa, synthetic construct
AQKVTQAQTEISVVEKEDVTLDCVYETRDQNYYLFWYKQPPSGELVFLIRRNSFDEQNEISGRYSWNFQKSTSSFNFTITASQVVDSAVYFCALSEAGNDMRFGAGTRLTVKP

SEQ ID NO: 16 - protein, 113 aa, synthetic construct
AQKVTQAQTEISVVEKEDVTLDCVYETRDENFYLFWYKQPPSGELVFLIRRNSFDEQNEISGRYSWNFQKSTSSFNFTITASQVVDSAVYFCALSEAGNDMRFGAGTRLTVKP

SEQ ID NO: 17 - protein, 113 aa, synthetic construct
AQKVTQAQTEISVVEKEDVTLDCVYETRDENYYLFWYKQPPSGELVFLIRRNSFDEQNEISGRYSWNFQKSTSSFNFTITASQVVDSAVYFCALSEAGNDMRFGAGTRLTVKP

SEQ ID NO: 18 - protein, 113 aa, synthetic construct
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEAGRITNFGAGTRLTVKP

SEQ ID NO: 19 - protein, 113 aa, synthetic construct
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEAGRMLHFGAGTRLTVKP

SEQ ID NO: 20 - protein, 113 aa, synthetic construct
AQKVTQAQTEISVVEKEDVTLDCVYETRDTTYYLFWYKQPPSGELVFLIRRNSFDEQNEISGRYSWNFQKSTSSFNFTITASQVVDSAVYFCALIEAGRTQAFGAGTRLTVKP

SEQ ID NO: 21 - protein, 113 aa, synthetic construct
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEAGRNTQFGAGTRLTVKP

SEQ ID NO: 22 - protein, 113 aa, synthetic construct
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEAGRQLKFGAGTRLTVKP

SEQ ID NO: 23 - protein, 113 aa, synthetic construct
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEAGRTTHFGAGTRLTVKP

SEQ ID NO: 24 - protein, 113 aa, synthetic construct
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEAGRTKQFGAGTRLTVKP

SEQ ID NO: 25 - protein, 113 aa, synthetic construct
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEAGRNLAFGAGTRLTVKP

SEQ ID NO: 26 - protein, 113 aa, synthetic construct
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEAGRNKSFGAGTRLTVKP

SEQ ID NO: 27 - protein, 113 aa, synthetic construct
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEAGRLTHFGAGTRLTVKP

SEQ ID NO: 28 - protein, 113 aa, synthetic construct
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEAGPLHSFGAGTRLTVKP

SEQ ID NO: 29 - protein, 113 aa, synthetic construct
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEAGRQLSFGAGTRLTVKP

SEQ ID NO: 30 - protein, 113 aa, synthetic construct
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEAGRMKKFGAGTRLTVKP

SEQ ID NO: 31 - protein, 113 aa, synthetic construct
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEAGRQKTFGAGTRLTVKP

SEQ ID NO: 32 - protein, 113 aa, synthetic construct
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEAGRLTAFGAGTRLTVKP

SEQ ID NO: 33 - protein, 113 aa, synthetic construct
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEAGRLLTFGAGTRLTVKP

SEQ ID NO: 34 - protein, 113 aa, synthetic construct
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEAGRQQTFGAGTRLTVKP

SEQ ID NO: 35 - protein, 113 aa, synthetic construct
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEAGRSKNFGAGTRLTVKP

SEQ ID NO: 36 - protein, 113 aa, synthetic construct
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEAGRNTDFGAGTRLTVKP

SEQ ID NO: 37 - protein, 113 aa, synthetic construct
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEAGRQTVFGAGTRLTVKP

SEQ ID NO: 38 - protein, 113 aa, synthetic construct
AQKVTQAQTEISVVEKEDVTLDCVYETRDDNYYLFWYKQPPSGELVFLIRRNSFDEQNEISGRYSWNFQKSTSSFNFTITASQVVDSAVYFCALSEAGNDMRFGAGTRLTVKP

SEQ ID NO: 39 - protein, 113 aa, synthetic construct
AQKVTQAQTEISVVEKEDVTLDCVYETRDMNYYLFWYKQPPSGELVFLIRRNSFDEQNEISGRYSWNFQKSTSSFNFTITASQVVDSAVYFCALSEAGNDMRFGAGTRLTVKP

SEQ ID NO: 40 - protein, 113 aa, synthetic construct
AQKVTQAQTEISVVEKEDVTLDCVYETRDSNYYLFWYKQPPSGELVFLIRRNSFDEQNEISGRYSWNFQKSTSSFNFTITASQVVDSAVYFCALSEAGNDMRFGAGTRLTVKP

SEQ ID NO: 41 - protein, 113 aa, synthetic construct
AQKVTQAQTEISVVEKEDVTLDCVYETRDVNYYLFWYKQPPSGELVFLIRRNSFDEQNEISGRYSWNFQKSTSSFNFTITASQVVDSAVYFCALSEAGNDMRFGAGTRLTVKP

SEQ ID NO: 42 - protein, 113 aa, synthetic construct
AQKVTQAQTEISVVEKEDVTLDCVYETRDYNYYLFWYKQPPSGELVFLIRRNSFDEQNEISGRYSWNFQKSTSSFNFTITASQVVDSAVYFCALSEAGNDMRFGAGTRLTVKP

SEQ ID NO: 43 - protein, 113 aa, synthetic construct
AQKVTQAQTEISVVEKEDVTLDCVYETRDTTYYLFWYKQPPSGELVFLIRQTSYDEQNEISGRYSWNFQKSTSSFNFTITASQVVDSAVYFCALSEAGNDMRFGAGTRLTVKP

SEQ ID NO: 44 - protein, 113 aa, synthetic construct
AQKVTQAQTEISVVEKEDVTLDCVYETRDTTYYLFWYKQPPSGELVFLIRQVSFDEQNEISGRYSWNFQKSTSSFNFTITASQVVDSAVYFCALSEAGNDMRFGAGTRLTVKP

SEQ ID NO: 45 - protein, 113 aa, synthetic construct
AQKVTQAQTEISVVEKEDVTLDCVYETRDTTYYLFWYKQPPSGELVFLIRRQSWDEQNEISGRYSWNFQKSTSSFNFTITASQVVDSAVYFCALSEAGNDMRFGAGTRLTVKP

SEQ ID NO: 46 - protein, 113 aa, synthetic construct
AQKVTQAQTEISVVEKEDVTLDCVYETRDTTYYLFWYKQPPSGELVFLIRRTSWDEQNEISGRYSWNFQKSTSSFNFTITASQVVDSAVYFCALSEAGNDMRFGAGTRLTVKP

SEQ ID NO: 47 - protein, 112 aa, synthetic construct
NAGVTQTPKFRVLKTGQSMTLLCAQDMNHEYMYWYRQDPGMGLRLIHYSVHEGTTAKGEVPDGYNVSRLKKQNFLLGLESAAPSQTSVYFCASSYLWSYEQYFGPGTRLTVT

SEQ ID NO: 48 - protein, 112 aa, synthetic construct
NAGVTQTPKFRVLKTGQSMTLLCAQDMNHEYMYWYRQDPGMGLRLIHYSVGEETTAKGEVPDGYNVSRLKKQNFLLGLESAAPSQTSVYFCASSYLWSYEQYFGPGTRLTVT

SEQ ID NO: 49 - protein, 112 aa, synthetic construct
NAGVTQTPKFRVLKTGQSMTLLCAQDMNHEYMYWYRQDPGMGLRLIHYSVGEHTTAKGEVPDGYNVSRLKKQNFLLGLESAAPSQTSVYFCASSYLWSYEQYFGPGTRLTVT

SEQ ID NO: 50 - protein, 112 aa, synthetic construct
NAGVTQTPKFRVLKTGQSMTLLCAQDMNHEYMYWYRQDPGMGLRLIHYSVGEGHTAKGEVPDGYNVSRLKKQNFLLGLESAAPSQTSVYFCASSYLWSYEQYFGPGTRLTVT

SEQ ID NO: 51 - protein, 112 aa, synthetic construct
NAGVTQTPKFRVLKTGQSMTLLCAQDMNHEYMYWYRQDPGMGLRLIHYSVGEDTTAKGEVPDGYNVSRLKKQNFLLGLESAAPSQTSVYFCASSYLWSYEQYFGPGTRLTVT

SEQ ID NO: 52 - protein, 112 aa, synthetic construct
NAGVTQTPKFRVLKTGQSMTLLCAQDMNHEYMYWYRQDPGMGLRLIHYSVGENTTAKGEVPDGYNVSRLKKQNFLLGLESAAPSQTSVYFCASSYLWSYEQYFGPGTRLTVT

SEQ ID NO: 53 - protein, 112 aa, synthetic construct
NAGVTQTPKFRVLKTGQSMTLLCAQDMNHEYMYWYRQDPGMGLRLIHYSVGEQTTAKGEVPDGYNVSRLKKQNFLLGLESAAPSQTSVYFCASSYLWSYEQYFGPGTRLTVT

SEQ ID NO: 54 - protein, 112 aa, synthetic construct
NAGVTQTPKFRVLKTGQSMTLLCAQDMNHEYMYWYRQDPGMGLRLIHYSVGEKTTAKGEVPDGYNVSRLKKQNFLLGLESAAPSQTSVYFCASSYLWSYEQYFGPGTRLTVT

SEQ ID NO: 55 - protein, 112 aa, synthetic construct
NAGVTQTPKFRVLKTGQSMTLLCAQDMNHEYMYWYRQDPGMGLRLIHYSVGERTTAKGEVPDGYNVSRLKKQNFLLGLESAAPSQTSVYFCASSYLWSYEQYFGPGTRLTVT

SEQ ID NO: 56 - protein, 112 aa, synthetic construct
NAGVTQTPKFRVLKTGQSMTLLCAQDMNHEYMYWYRQDPGMGLRLIHYSVGQHSTAKGEVPDGYNVSRLKKQNFLLGLESAAPSQTSVYFCASSYLWSYEQYFGPGTRLTVT

SEQ ID NO: 57 - protein, 112 aa, synthetic construct
NAGVTQTPKFRVLKTGQSMTLLCAQDMNHEYMYWYRQDPGMGLRLIHYSVGNTITAKGEVPDGYNVSRLKKQNFLLGLESAAPSQTSVYFCASSYLWSYEQYFGPGTRLTVT

SEQ ID NO: 58 - protein, 112 aa, synthetic construct
NAGVTQTPKFRVLKTGQSMTLLCAQDMNHEYMYWYRQDPGMGLRLIHYSVGKEITAKGEVPDGYNVSRLKKQNFLLGLESAAPSQTSVYFCASSYLWSYEQYFGPGTRLTVT

SEQ ID NO: 59 - protein, 112 aa, synthetic construct
NAGVTQTPKFRVLKTGQSMTLLCAQDMNHEYMYWYRQDPGMGLRLIHYSVGTTTTAKGEVPDGYNVSRLKKQNFLLGLESAAPSQTSVYFCASSYLWSYEQYFGPGTRLTVT

SEQ ID NO: 60 - protein, 112 aa, synthetic construct
NAGVTQTPKFRVLKTGQSMTLLCAQDNNHDYMYWYRQDPGMGLRLIHYSVGEGTTAKGEVPDGYNVSRLKKQNFLLGLESAAPSQTSVYFCASSYLWSYEQYFGPGTRLTVT

SEQ ID NO: 61 - protein, 207 aa, synthetic construct
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEAGNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 62 - protein, 242 aa, synthetic construct
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSYLWSYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRAD

SEQ ID NO: 63 - protein, 254 aa, synthetic construct
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEAGNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 64 - protein, 291 aa, synthetic construct
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSYLWSYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 65 - protein, 7 aa, synthetic construct
TRDTTYY

SEQ ID NO: 66 - protein, 8 aa, synthetic construct
RNSFDEQN

SEQ ID NO: 67 - protein, 10 aa, synthetic construct
ALSEAGNDMR

SEQ ID NO: 68 - protein, 7 aa, synthetic construct
TRDTDYY

SEQ ID NO: 69 - protein, 7 aa, synthetic construct
TRDTNYY

SEQ ID NO: 70 - protein, 7 aa, synthetic construct
TRDQNYY

SEQ ID NO: 71 - protein, 7 aa, synthetic construct
TRDENFY

SEQ ID NO: 72 - protein, 7 aa, synthetic construct
TRDENYY

SEQ ID NO: 73 - protein, 10 aa, synthetic construct
ALSEAGRITN

SEQ ID NO: 74 - protein, 10 aa, synthetic construct
ALSEAGRMLH

SEQ ID NO: 75 - protein, 10 aa, synthetic construct
ALIEAGRTQA

SEQ ID NO: 76 - protein, 10 aa, synthetic construct
ALSEAGRNTQ

SEQ ID NO: 77 - protein, 10 aa, synthetic construct
ALSEAGRQLK

SEQ ID NO: 78 - protein, 10 aa, synthetic construct
ALSEAGRTTH

SEQ ID NO: 79 - protein, 10 aa, synthetic construct
ALSEAGRTKQ

SEQ ID NO: 80 - protein, 10 aa, synthetic construct
ALSEAGRNLA

SEQ ID NO: 81 - protein, 10 aa, synthetic construct
ALSEAGRNKS

SEQ ID NO: 82 - protein, 10 aa, synthetic construct
ALSEAGRLTH

SEQ ID NO: 83 - protein, 10 aa, synthetic construct
ALSEAGPLHS

SEQ ID NO: 84 - protein, 10 aa, synthetic construct
ALSEAGRQLS

SEQ ID NO: 85 - protein, 10 aa, synthetic construct
ALSEAGRMKK

SEQ ID NO: 86 - protein, 10 aa, synthetic construct
ALSEAGRQKT

SEQ ID NO: 87 - protein, 10 aa, synthetic construct
ALSEAGRLTA

SEQ ID NO: 88 - protein, 10 aa, synthetic construct
ALSEAGRLLT

SEQ ID NO: 89 - protein, 10 aa, synthetic construct
ALSEAGRQQT

SEQ ID NO: 90 - protein, 10 aa, synthetic construct
ALSEAGRSKN

SEQ ID NO: 91 - protein, 10 aa, synthetic construct
ALSEAGRNTD

SEQ ID NO: 92 - protein, 10 aa, synthetic construct
ALSEAGRQTV

SEQ ID NO: 93 - protein, 7 aa, synthetic construct
TRDDNYY

SEQ ID NO: 94 - protein, 7 aa, synthetic construct
TRDMNYY

SEQ ID NO: 95 - protein, 7 aa, synthetic construct
TRDSNYY

SEQ ID NO: 96 - protein, 7 aa, synthetic construct
TRDVNYY

SEQ ID NO: 97 - protein, 7 aa, synthetic construct
TRDYNYY

SEQ ID NO: 98 - protein, 8 aa, synthetic construct
QTSYDEQN

SEQ ID NO: 99 - protein, 8 aa, synthetic construct
QVSFDEQN

SEQ ID NO: 100 - protein, 8 aa, synthetic construct
RQSWDEQN

SEQ ID NO: 101 - protein, 8 aa, synthetic construct
RTSWDEQN

SEQ ID NO: 102 - protein, 6 aa, synthetic construct
SVHEGT

SEQ ID NO: 103 - protein, 6 aa, synthetic construct
SVGEET

SEQ ID NO: 104 - protein, 6 aa, synthetic construct
SVGEHT

SEQ ID NO: 105 - protein, 6 aa, synthetic construct
SVGEGH

SEQ ID NO: 106 - protein, 6 aa, synthetic construct
SVGEDT

SEQ ID NO: 107 - protein, 6 aa, synthetic construct
SVGENT

SEQ ID NO: 108 - protein, 6 aa, synthetic construct
SVGEQT

SEQ ID NO: 109 - protein, 6 aa, synthetic construct
SVGEKT

SEQ ID NO: 110 - protein, 6 aa, synthetic construct
SVGERT

SEQ ID NO: 111 - protein, 6 aa, synthetic construct
SVGQHS

SEQ ID NO: 112 - protein, 6 aa, synthetic construct
SVGNTI

SEQ ID NO: 113 - protein, 6 aa, synthetic construct
SVGKEI

SEQ ID NO: 114 - protein, 6 aa, synthetic construct
SVGTTT

SEQ ID NO: 115 - protein, 5 aa, synthetic construct
NNHDY

SEQ ID NO: 116 - protein, 5 aa, synthetic construct
MNHEY

SEQ ID NO: 117 - protein, 6 aa, synthetic construct
SVGEGT

SEQ ID NO: 118 - protein, 11 aa, synthetic construct
ASSYLWSYEQY

(19) United States
(12) Patent Application Publication
(10) Pub. No.: US 2026/0297159 A1
(43) Pub. Date: Oct. 1, 2026

(54) HIGH-AFFINITY T CELL RECEPTOR FOR IDENTIFYING KRAS MUTATION AND USE THEREOF

(71) Applicant: XLIFESC, LTD., Guangzhou, Guangdong (CN)

(72) Inventors: Yushuang LIANG, Guangzhou, Guangdong (CN); Kai ZHAN, Guangzhou, Guangdong (CN); Zhen PENG, Guangzhou, Guangdong (CN); Hanli SUN, Guangzhou, Guangdong (CN); Cuiqiong ZHANG, Guangzhou, Guangdong (CN); Jiao HUANG, Guangzhou, Guangdong (CN); Zhiming WENG, Guangzhou, Guangdong (CN); Yingyi MEI, Guangzhou, Guangdong (CN)

(21) Appl. No.: 19/476,785

(22) PCT Filed: Apr. 19, 2024
(86) PCT No.: PCT/CN2024/088802

§ 371 Date: Oct. 20, 2025

(30) Foreign Application Priority Data
Apr. 19, 2023 (CN) 202310422033.4

(51) Int. Cl. C07K 14/705; A61K 35/17; A61K 38/00; A61K 40/11; A61K 40/32; A61K 40/42; A61K 49/00; A61P 35/00; C07K 16/28; C07K 19/00; C12N 5/0783; C12N 15/63

(52) U.S. Cl. CPC C07K 14/7051; A61K 35/17; A61K 38/00; A61K 40/11; A61K 40/32; A61K 40/42; A61K 49/00; A61P 35/00; C07K 14/70517; C07K 16/2833; C07K 19/00; C12N 5/0636; C12N 15/63; A61K 2123/00; C07K 2317/35; C07K 2317/565; C07K 2319/03; C12N 2510/00

(57) ABSTRACT

Provided is a TCR having the characteristics of binding to a VVVGADGVGK-HLA A1101 complex. Also provided are a multivalent TCR complex, a nucleic acid molecule encoding the TCR, a vector comprising the nucleic acid molecule, a cell expressing the TCR, and a pharmaceutical composition comprising the described substance, which can be used for preparing a medicament for diagnosing, treating, and preventing a KRAS G12D positive disease. Also provided is a preparation method for the TCR.

### RELATED APPLICATIONS

The present application claims the priority of a Chinese patent application filed on Apr. 19, 2023, with an application No. 2023104220334 and the title “A High-Affinity T Cell Receptor for identifying KRAS Mutation and use thereof”, the entire text of which is incorporated herein by reference.

### REFERENCE TO THE SEQUENCE LISTING

The specification is submitted along with a Sequence Listing. The file name of the Sequence Listing is sequence_listing.xml, with a size of 111,864 bytes, created on Oct. 17, 2025; and the entire contents thereof are incorporated herein by reference.

### TECHNICAL FIELD

The present application belongs to the field of biomedicine and biotechnology, and relates to a high-affinity T cell receptor that identifies KRAS G12D and uses thereof; and more specifically, it relates to a T cell receptor (TCR) that can recognize peptides derived from the KRAS G12D protein; and the present application also relates to a preparation method and use of said TCR.

### BACKGROUND

Only two types of molecules can recognize antigens in a specific manner. One of them is the immunoglobulin or antibody; and the other is the T cell receptor (TCR), which is a glycoprotein on the cell membrane existing as a heterodimer composed of α/β chains or γ/δ chains. The composition of the total TCR repertoire in the immune system is generated in the thymus through V(D)J recombination, followed by positive and negative selection. In the peripheral environment, TCR mediates the specific recognition of major histocompatibility complex-peptide complexes (pMHC) by T cells, so it is crucial for the cellular immune function of the immune system.

TCR is the only receptor for specific antigenic peptides presented on the major histocompatibility complex (MHC), and such exogenous or endogenous peptides may be the only sign of cellular abnormalities. In the immune system, the binding of antigen-specific TCR to the pMHC complex triggers direct physical contact between T cells and antigen-presenting cells (APCs). Subsequently, other cell membrane surface molecules of both T cells and APCs interact with each other, which induces a series of subsequent cellular signal transduction and other physiological responses, thereby enabling T cells with different antigen specificities to exert immune effects on their target cells.

The MHC class I and class II molecule ligands corresponding to TCR are also proteins of the immunoglobulin superfamily, but they are specific for antigen presentation. Different individuals have different MHCs, which can thus present different short peptides from a protein antigen to the surface of their respective APC cells. The human MHC is usually referred to as the HLA gene or HLA complex.

The KRAS gene (P21 gene) is a murine sarcoma virus oncogene and a member of the ras gene family, encoding the KRAS protein. Once the KRAS gene mutates, it will continuously stimulate cell growth, leading to the occurrence of tumors. Among them, G12D is one of the common mutation sites, and this mutant is called KRAS G12D. KRAS G12D is expressed in various human cancer cells, including but not limited to lung cancer, colorectal cancer, pancreatic cancer, gastric cancer, etc., as reported in literature (FISHER G H et al (2001) Genes Dev 15(24):3249-3262; Brychta N et al (2016) Clin Chem 62(11):1482-1491; Ondrej Fiala et al (2016) Tumour Biol 37(5):6823-30; Ana S. Leal et al (2018) Curr Protoc Pharmacol 83(1):e48, etc.). The short peptide VVVGADGVGK from KRAS G12D mutation is located at amino acids 7-16 of KRAS and is a target for treating related diseases. After KRAS G12D is produced in cells, it is degraded into small molecular polypeptides, which combine with MHC (major histocompatibility complex) molecules to form complexes and are presented on the cell surface. VVVGADGVGK is a short peptide derived from the KRAS G12D antigen. For treating the above-mentioned diseases, methods such as chemotherapy and radiotherapy can be used, but they will cause damage to one's own normal cells.

Therefore, the VVVGADGVGK-HLA A1101 complex provides a marker on tumor cells that can be targeted by TCRs. TCRs capable of binding to the VVVGADGVGK-HLA A1101 complex have high application value in tumor treatment. For example, TCRs that can target this tumor cell marker can be used to deliver cytotoxic agents or immunostimulants to target cells, or be transferred into T cells, enabling T cells expressing such TCRs to destroy tumor cells for the administration to patients in a therapeutic process known as adoptive immunotherapy. For the former purpose, ideal TCRs should have high affinity, allowing them to remain on the targeted cells for a long time. For the latter purpose, TCRs with moderate affinity are preferred. Therefore, a skilled person is committed to developing TCRs that target tumor cell markers to meet different purposes.

### SUMMARY OF THE INVENTION

In view of the shortcomings of traditional technologies, the purpose of the present application is to provide a high-affinity T cell receptor for identifying KRAS mutations and uses thereof. The technical solution is as follows:

In the first aspect of the present application, a T cell receptor (TCR) is provided, which comprises a TCRα chain variable domain and a TCRβ chain variable domain, and the TCR has the activity of binding to the VVVGADGVGK-HLA A1101 complex;
and the amino acid sequence of the TCRα chain variable domain has at least 90% (for example, it can be 90%, 91%, 92%, 93%, 94%, 95%, 96%, 97%, 98%, 99%, or 100%, etc.) sequence homology with the amino acid sequence shown in SEQ ID NO: 1, and the amino acid sequence of the TCRβ chain variable domain has at least 90% (for example, it can be 90%, 91%, 92%, 93%, 94%, 95%, 96%, 97%, 98%, 99%, or 100%, etc.) sequence homology with the amino acid sequence shown in SEQ ID NO: 2.

SEQ ID NO: 1
AQKVTQAQTEISVVEKEDVTLDCVYETRDTTYYLFWYKQP
PSGELVFLIRRNSFDEQNEISGRYSWNFQKSTSSFNFTIT
ASQVVDSAVYFCALSEAGNDMRFGAGTRLTVKP.
SEQ ID NO: 2
NAGVTQTPKFRVLKTGQSMTLLCAQDMNHEYMYWYRQDPG
MGLRLIHYSVGEGTTAKGEVPDGYNVSRLKKQNFLLGLES
AAPSQTSVYFCASSYLWSYEQYFGPGTRLTVT.

In one example, the amino acid sequence of the TCRα chain variable domain and the amino acid sequence of the TCRβ chain variable domain are not both the amino acid sequence of the wild-type TCRα chain variable domain and the amino acid sequence of the wild-type TCRβ chain variable domain.

In another example, the TCR α chain variable domain comprises an amino acid sequence having at least 91%, 92%, 93%, 94%, 95%, 96%, 97%, 98%, or 99% sequence homology with the sequence shown in SEQ ID NO: 1, or comprises 1, 2, 3, 4, 5, 6, 7, 8, 9, 10, or 11 amino acid residue mutations as compared with the sequence shown in SEQ ID NO: 1.

In another example, the TCR β chain variable domain is an amino acid sequence having at least 91%, 92%, 93%, 94%, 95%, 96%, 97%, 98%, 99% or 100% sequence homology with the sequence shown in SEQ ID NO: 2, or comprises 1, 2, 3, 4, 5, 6, 7, 8, 9, 10 or 11 amino acid residue mutations as compared with the sequence shown in SEQ ID NO: 2.

In another example, the amino acid sequence of the TCRα chain variable domain has at least 95% sequence homology with the amino acid sequence shown in SEQ ID NO: 1, and the amino acid sequence of the TCRβ chain variable domain has at least 95% sequence homology with the amino acid sequence shown in SEQ ID NO: 2.

In another example, in the TCRβ chain variable domain, the CDR1β is MNHEY, the CDR2β is SVGEGT, and the CDR3β is ASSYLWSYEQY.

In another example, the TCR also has the activity of binding to VVVGADGVGK-HLA A1101 complex.

In another example, the three CDRs of the TCRα chain variable domain are:

CDR1α:
SEQ ID NO: 65
TRDTTYY;
CDR2α:
SEQ ID NO: 66
RNSFDEQN;
and,
CDR3α:
SEQ ID NO: 67
ALSEAGNDMR;

and comprises at least one mutation from Table 1:

TABLE 1

| Residue before mutation | Residue after mutation |
|---|---|
| T at position 4 | E or Q or D or M or S or V or Y |
| of CDR1α | |
| T at position 5 | N or D |
| of CDR1α | |
| Y at position 6 | F |
| of CDR1α | |
| R at position 1 | Q |
| of CDR2α | |
| N at position 2 | T or V or Q |
| of CDR2α | |
| F at position 4 | Y or W |
| of CDR2α | |
| N at position 7 | R |
| of CDR3α | |
| D at position 8 | I or M or T or N or Q or L or S |
| of CDR3α | |
| M at position 9 | T or L or Q or K or H |
| of CDR3α | |
| R at position 10 | N or H or A or Q or K or S or T or D or V |
| of CDR3α | |

In another example, the amino acid sequence of the TCRβ chain variable domain is SEQ ID NO: 2.

In another example, the three CDRs of the TCRβ chain variable domain are:

CDRIβ:
SEQ ID NO: 116
MNHEY;
CDR2β:
SEQ ID NO: 117
SVGEGT;
and,
CDR3β:
SEQ ID NO: 118
ASSYLWSYEQY;

and comprises at least one mutation from Table 2:

TABLE 2

| Residue before mutation | Residue after mutation |
|---|---|
| M at position 1 | N |
| of CDR1β | |
| E at position 4 | D |
| of CDR1β | |
| G at position 3 | H |
| of CDR2β | |
| E at position 4 | Q or N or K or T |
| of CDR2β | |
| G at position 5 | E or H or D or N or Q or K or R or T |
| of CDR2β | |
| T at position 6 | H or S or I |
| of CDR2β | |

In another example, the affinity of the TCR for the VVVGADGVGK-HLA A1101 complex is at least twice that of the wild-type TCR, and can be, for example, 2, 2.5, 3, 3.5, 4, 4.5, or 5 times, etc.

In another example, the TCR has a mutation in the α chain variable domain shown in SEQ ID NO: 1, and the mutation is selected from one or more groups of T30E/Q/D/M/S/V/Y, T31N/D, Y32F, R51Q, N52T/V/Q, F54Y/W, N99R, D100I/M/T/N/Q/L/S, M101T/L/Q/K/H, and R102N/H/A/Q/K/S/T/D/V, wherein the numbering of amino acid residues adopts the numbering shown in SEQ ID NO: 1.

In another example, the TCR has a mutation in the β chain variable domain shown in SEQ ID NO: 2, and the mutation is selected from one or more groups of M27N, E30D, G51H, E52Q/N/K/T, G53E/H/D/N/Q/K/R/T, and T54H/S/I, wherein the numbering of amino acid residues adopts the numbering shown in SEQ ID NO: 2.

In another example, the TCR has CDRs selected from Table 3:

TABLE 3

| CDR | | | | | | |
|---|---|---|---|---|---|---|
| number | α-CDR1 | α-CDR2 | α-CDR3 | β-CDR1 | β-CDR2 | β-CDR3 |
| 1 | TRDTDYY | RNSFDEQN | ALSEAGNDMR | MNHEY | SVGEGT | ASSYLWSYEQY |
| 2 | TRDTNYY | RNSFDEQN | ALSEAGNDMR | MNHEY | SVGEGT | ASSYLWSYEQY |
| 3 | TRDQNYY | RNSFDEQN | ALSEAGNDMR | MNHEY | SVGEGT | ASSYLWSYEQY |
| 4 | TRDENFY | RNSFDEQN | ALSEAGNDMR | MNHEY | SVGEGT | ASSYLWSYEQY |
| 5 | TRDENYY | RNSFDEQN | ALSEAGNDMR | MNHEY | SVGEGT | ASSYLWSYEQY |
| 6 | TRDTTYY | RNSFDEQN | ALSEAGRITN | MNHEY | SVGEGT | ASSYLWSYEQY |
| 7 | TRDTTYY | RNSFDEQN | ALSEAGRMLH | MNHEY | SVGEGT | ASSYLWSYEQY |
| 8 | TRDTTYY | RNSFDEQN | ALIEAGRTQA | MNHEY | SVGEGT | ASSYLWSYEQY |
| 9 | TRDTTYY | RNSFDEQN | ALSEAGRNTQ | MNHEY | SVGEGT | ASSYLWSYEQY |
| 10 | TRDTTYY | RNSFDEQN | ALSEAGRQLK | MNHEY | SVGEGT | ASSYLWSYEQY |
| 11 | TRDTTYY | RNSFDEQN | ALSEAGRTTH | MNHEY | SVGEGT | ASSYLWSYEQY |
| 12 | TRDTTYY | RNSFDEQN | ALSEAGRTKQ | MNHEY | SVGEGT | ASSYLWSYEQY |
| 13 | TRDTTYY | RNSFDEQN | ALSEAGRNLA | MNHEY | SVGEGT | ASSYLWSYEQY |
| 14 | TRDTTYY | RNSFDEQN | ALSEAGRNKS | MNHEY | SVGEGT | ASSYLWSYEQY |
| 15 | TRDTTYY | RNSFDEQN | ALSEAGRLTH | MNHEY | SVGEGT | ASSYLWSYEQY |
| 16 | TRDTTYY | RNSFDEQN | ALSEAGPLHS | MNHEY | SVGEGT | ASSYLWSYEQY |
| 17 | TRDTTYY | RNSFDEQN | ALSEAGRQLS | MNHEY | SVGEGT | ASSYLWSYEQY |
| 18 | TRDTTYY | RNSFDEQN | ALSEAGRMKK | MNHEY | SVGEGT | ASSYLWSYEQY |
| 19 | TRDTTYY | RNSFDEQN | ALSEAGRQKT | MNHEY | SVGEGT | ASSYLWSYEQY |
| 20 | TRDTTYY | RNSFDEQN | ALSEAGRLTA | MNHEY | SVGEGT | ASSYLWSYEQY |
| 21 | TRDTTYY | RNSFDEQN | ALSEAGRLLT | MNHEY | SVGEGT | ASSYLWSYEQY |
| 22 | TRDTTYY | RNSFDEQN | ALSEAGRQQT | MNHEY | SVGEGT | ASSYLWSYEQY |
| 23 | TRDTTYY | RNSFDEQN | ALSEAGRSKN | MNHEY | SVGEGT | ASSYLWSYEQY |
| 24 | TRDTTYY | RNSFDEQN | ALSEAGRNTD | MNHEY | SVGEGT | ASSYLWSYEQY |
| 25 | TRDTTYY | RNSFDEQN | ALSEAGRQTV | MNHEY | SVGEGT | ASSYLWSYEQY |
| 26 | TRDDNYY | RNSFDEQN | ALSEAGNDMR | MNHEY | SVGEGT | ASSYLWSYEQY |
| 27 | TRDMNYY | RNSFDEQN | ALSEAGNDMR | MNHEY | SVGEGT | ASSYLWSYEQY |
| 28 | TRDSNYY | RNSFDEQN | ALSEAGNDMR | MNHEY | SVGEGT | ASSYLWSYEQY |
| 29 | TRDVNYY | RNSFDEQN | ALSEAGNDMR | MNHEY | SVGEGT | ASSYLWSYEQY |
| 30 | TRDYNYY | RNSFDEQN | ALSEAGNDMR | MNHEY | SVGEGT | ASSYLWSYEQY |
| 31 | TRDTTYY | QTSYDEQN | ALSEAGNDMR | MNHEY | SVGEGT | ASSYLWSYEQY |
| 32 | TRDTTYY | QVSFDEQN | ALSEAGNDMR | MNHEY | SVGEGT | ASSYLWSYEQY |
| 33 | TRDTTYY | RQSWDEQN | ALSEAGNDMR | MNHEY | SVGEGT | ASSYLWSYEQY |
| 34 | TRDTTYY | RTSWDEQN | ALSEAGNDMR | MNHEY | SVGEGT | ASSYLWSYEQY |
| 35 | TRDTTYY | RNSFDEQN | ALSEAGNDMR | MNHEY | SVHEGT | ASSYLWSYEQY |
| 36 | TRDTTYY | RNSFDEQN | ALSEAGNDMR | MNHEY | SVGEET | ASSYLWSYEQY |
| 37 | TRDTTYY | RNSFDEQN | ALSEAGNDMR | MNHEY | SVGEHT | ASSYLWSYEQY |
| 38 | TRDTTYY | RNSFDEQN | ALSEAGNDMR | MNHEY | SVGEGH | ASSYLWSYEQY |
| 39 | TRDTTYY | RNSFDEQN | ALSEAGNDMR | MNHEY | SVGEDT | ASSYLWSYEQY |
| 40 | TRDTTYY | RNSFDEQN | ALSEAGNDMR | MNHEY | SVGENT | ASSYLWSYEQY |
| 41 | TRDTTYY | RNSFDEQN | ALSEAGNDMR | MNHEY | SVGEQT | ASSYLWSYEQY |
| 42 | TRDTTYY | RNSFDEQN | ALSEAGNDMR | MNHEY | SVGEKT | ASSYLWSYEQY |
| 43 | TRDTTYY | RNSFDEQN | ALSEAGNDMR | MNHEY | SVGERT | ASSYLWSYEQY |
| 44 | TRDTTYY | RNSFDEQN | ALSEAGNDMR | MNHEY | SVGQHS | ASSYLWSYEQY |
| 45 | TRDTTYY | RNSFDEQN | ALSEAGNDMR | MNHEY | SVGNTI | ASSYLWSYEQY |
| 46 | TRDTTYY | RNSFDEQN | ALSEAGNDMR | MNHEY | SVGKEI | ASSYLWSYEQY |
| 47 | TRDTTYY | RNSFDEQN | ALSEAGNDMR | MNHEY | SVGTTT | ASSYLWSYEQY |
| 48 | TRDTTYY | RNSFDEQN | ALSEAGNDMR | NNHDY | SVGEGT | ASSYLWSYEQY |

Amino acid sequences of SEQ ID NO: 68-
SEQ ID NO: 115 are shown as follows:
SEQ ID NO: 68
TRDTDYY
SEQ ID NO: 69
TRDTNYY
SEQ ID NO: 70
TRDQNYY
SEQ ID NO: 71
TRDENFY
SEQ ID NO: 72
TRDENYY
SEQ ID NO: 73
ALSEAGRITN
SEQ ID NO: 74
ALSEAGRMLH
SEQ ID NO: 75
ALIEAGRTQA
SEQ ID NO: 76
ALSEAGRNTQ
SEQ ID NO: 77
ALSEAGRQLK
SEQ ID NO: 78
ALSEAGRTTH
SEQ ID NO: 79
ALSEAGRTKQ
SEQ ID NO: 80
ALSEAGRNLA
SEQ ID NO: 81
ALSEAGRNKS
SEQ ID NO: 82
ALSEAGRLTH
SEQ ID NO: 83
ALSEAGPLHS
SEQ ID NO: 84
ALSEAGRQLS
SEQ ID NO: 85
ALSEAGRMKK
SEQ ID NO: 86
ALSEAGRQKT
SEQ ID NO: 87
ALSEAGRLTA
SEQ ID NO: 88
ALSEAGRLLT
SEQ ID NO: 89
ALSEAGRQQT
SEQ ID NO: 90
ALSEAGRSKN
SEQ ID NO: 91
ALSEAGRNTD
SEQ ID NO: 92
ALSEAGRQTV
SEQ ID NO: 93
TRDDNYY
SEQ ID NO: 94
TRDMNYY
SEQ ID NO: 95
TRDSNYY
SEQ ID NO: 96
TRDVNYY
SEQ ID NO: 97
TRDYNYY
SEQ ID NO: 98
QTSYDEQN
SEQ ID NO: 99
QVSFDEQN
SEQ ID NO: 100
RQSWDEQN
SEQ ID NO: 101
RTSWDEQN
SEQ ID NO: 102
SVHEGT
SEQ ID NO: 103
SVGEET
SEQ ID NO: 104
SVGEHT
SEQ ID NO: 105
SVGEGH
SEQ ID NO: 106
SVGEDT
SEQ ID NO: 107
SVGENT
SEQ ID NO: 108
SVGEQT
SEQ ID NO: 109
SVGEKT
SEQ ID NO: 110
SVGERT
SEQ ID NO: 111
SVGQHS
SEQ ID NO: 112
SVGNTI
SEQ ID NO: 113
SVGKEI
SEQ ID NO: 114
SVGTTT
SEQ ID NO: 115
NNHDY

In another example, the TCR is soluble.

In another example, the TCR is an αβ heterodimeric TCR, and the αβ heterodimeric TCR comprises the α chain TRAC constant region sequence and the β chain TRBC1 or TRBC2 constant region sequence.

In another example, the TCR comprises (i) TCR α chain variable domain and all or part of the TCR α chain constant region except for transmembrane domain, and (ii) TCR β chain variable domain and all or part of the TCR β chain constant region except for transmembrane domain.

In another example, the TCR comprises α chain constant region and β chain constant region, and an artificial interchain disulfide bond is contained between the α chain constant region and the β chain constant region of the TCR; and optionally, cysteine residues forming an artificial interchain disulfide bond between the α chain constant region and the β chain constant region of the TCR are substituted for one or more groups of amino acids selected from the following:
Thr48 in exon 1 of TRAC*01 and Ser57 in exon 1 of TRBC1*01 or TRBC2*01;
Thr45 in exon 1 of TRAC*01 and Ser77 in exon 1 of TRBC1*01 or TRBC2*01;
Tyr10 in exon 1 of TRAC*01 and Ser17 in exon 1 of TRBC1*01 or TRBC2*01;
Thr45 in exon 1 of TRAC*01 and Asp59 in exon 1 of TRBC1*01 or TRBC2*01;
Ser15 in exon 1 of TRAC*01 and Glu15 in exon 1 of TRBC1*01 or TRBC2*01;
Arg53 in exon 1 of TRAC*01 and Ser54 in exon 1 of TRBC1*01 or TRBC2*01;
Pro89 in exon 1 of TRAC*01 and Ala19 in exon 1 of TRBC1*01 or TRBC2*01; and,
or Tyr10 in exon 1 of TRAC*01 and Glu20 in exon 1 of TRBC1*01 or TRBC2*01.

In another example, the amino acid sequence of the TCR α chain variable domain is selected from SEQ ID NO: 1, SEQ ID NO: 13-46; and/or the amino acid sequence of the TCR β chain variable domain is selected from SEQ ID NO: 2, SEQ ID NO: 47-60;

Optionally, the TCR is selected from the following group:
(1) the sequence of the α chain variable domain is SEQ ID NO:13, and the sequence of the β chain variable domain is SEQ ID NO:2
(2) the sequence of the α chain variable domain is SEQ ID NO:14, and the sequence of the β chain variable domain is SEQ ID NO:2
(3) the sequence of the α chain variable domain is SEQ ID NO:15, and the sequence of the β chain variable domain is SEQ ID NO:2
(4) the sequence of the α chain variable domain is SEQ ID NO:16, and the sequence of the β chain variable domain is SEQ ID NO:2
(5) the sequence of the α chain variable domain is SEQ ID NO:17, and the sequence of the β chain variable domain is SEQ ID NO:2
(6) the sequence of the α chain variable domain is SEQ ID NO:18, and the sequence of the β chain variable domain is SEQ ID NO: 2
(7) the sequence of the α chain variable domain is SEQ ID NO:19, and the sequence of the β chain variable domain is SEQ ID NO:2
(8) the sequence of the α chain variable domain is SEQ ID NO:20, and the sequence of the β chain variable domain is SEQ ID NO:2
(9) the sequence of the α chain variable domain is SEQ ID NO:21, and the sequence of the β chain variable domain is SEQ ID NO:2
(10) the sequence of the α chain variable domain is SEQ ID NO:22, and the sequence of the β chain variable domain is SEQ ID NO:2
(11) the sequence of the α chain variable domain is SEQ ID NO:23, and the sequence of the β chain variable domain is SEQ ID NO:2
(12) the sequence of the α chain variable domain is SEQ ID NO:24, and the sequence of the β chain variable domain is SEQ ID NO:2
(13) the sequence of the α chain variable domain is SEQ ID NO:25, and the sequence of the β chain variable domain is SEQ ID NO:2
(14) the sequence of the α chain variable domain is SEQ ID NO:26, and the sequence of the β chain variable domain is SEQ ID NO:2
(15) the sequence of the α chain variable domain is SEQ ID NO:27, and the sequence of the β chain variable domain is SEQ ID NO:2
(16) the sequence of the α chain variable domain is SEQ ID NO:28, and the sequence of the β chain variable domain is SEQ ID NO:2
(17) the sequence of the α chain variable domain is SEQ ID NO:29, and the sequence of the β chain variable domain is SEQ ID NO:2
(18) the sequence of the α chain variable domain is SEQ ID NO:30, and the sequence of the β chain variable domain is SEQ ID NO:2
(19) the sequence of the α chain variable domain is SEQ ID NO:31, and the sequence of the β chain variable domain is SEQ ID NO:2
(20) the sequence of the α chain variable domain is SEQ ID NO:32, and the sequence of the β chain variable domain is SEQ ID NO:2
(21) the sequence of the α chain variable domain is SEQ ID NO:33, and the sequence of the β chain variable domain is SEQ ID NO:2
(22) the sequence of the α chain variable domain is SEQ ID NO:34, and the sequence of the β chain variable domain is SEQ ID NO:2
(23) the sequence of the α chain variable domain is SEQ ID NO:35, and the sequence of the β chain variable domain is SEQ ID NO:2
(24) the sequence of the α chain variable domain is SEQ ID NO:36, and the sequence of the β chain variable domain is SEQ ID NO:2
(25) the sequence of the α chain variable domain is SEQ ID NO:37, and the sequence of the β chain variable domain is SEQ ID NO:2
(26) the sequence of the α chain variable domain is SEQ ID NO:38, and the sequence of the β chain variable domain is SEQ ID NO:2
(27) the sequence of the α chain variable domain is SEQ ID NO:39, and the sequence of the β chain variable domain is SEQ ID NO:2
(28) the sequence of the α chain variable domain is SEQ ID NO:40, and the sequence of the β chain variable domain is SEQ ID NO:2
(29) the sequence of the α chain variable domain is SEQ ID NO:41, and the sequence of the β chain variable domain is SEQ ID NO:2
(30) the sequence of the α chain variable domain is SEQ ID NO:42, and the sequence of the β chain variable domain is SEQ ID NO:2
(31) the sequence of the α chain variable domain is SEQ ID NO:43, and the sequence of the β chain variable domain is SEQ ID NO:2
(32) the sequence of the α chain variable domain is SEQ ID NO:44, and the sequence of the β chain variable domain is SEQ ID NO:2
(33) the sequence of the α chain variable domain is SEQ ID NO:45, and the sequence of the β chain variable domain is SEQ ID NO:2
(34) the sequence of the α chain variable domain is SEQ ID NO:46, and the sequence of the β chain variable domain is SEQ ID NO:2
(35) the sequence of the α chain variable domain is SEQ ID NO:1, and the sequence of the β chain variable domain is SEQ ID NO:47
(36) the sequence of the α chain variable domain is SEQ ID NO:1, and the sequence of the β chain variable domain is SEQ ID NO:48
(37) the sequence of the α chain variable domain is SEQ ID NO:1, and the sequence of the β chain variable domain is SEQ ID NO:49
(38) the sequence of the α chain variable domain is SEQ ID NO:1, and the sequence of the β chain variable domain is SEQ ID NO:50
(39) the sequence of the α chain variable domain is SEQ ID NO:1, and the sequence of the β chain variable domain is SEQ ID NO:51
(40) the sequence of the α chain variable domain is SEQ ID NO:1, and the sequence of the β chain variable domain is SEQ ID NO:52
(41) the sequence of the α chain variable domain is SEQ ID NO:1, and the sequence of the β chain variable domain is SEQ ID NO:53
(42) the sequence of the α chain variable domain is SEQ ID NO:1, and the sequence of the β chain variable domain is SEQ ID NO:54
(43) the sequence of the α chain variable domain is SEQ ID NO:1, and the sequence of the β chain variable domain is SEQ ID NO:55
(44) the sequence of the α chain variable domain is SEQ ID NO:1, and the sequence of the β chain variable domain is SEQ ID NO:56
(45) the sequence of the α chain variable domain is SEQ ID NO:1, and the sequence of the β chain variable domain is SEQ ID NO:57
(46) the sequence of the α chain variable domain is SEQ ID NO:1, and the sequence of the β chain variable domain is SEQ ID NO:58
(47) the sequence of the α chain variable domain is SEQ ID NO:1, and the sequence of the β chain variable domain is SEQ ID NO:59, and
(48) the sequence of the α chain variable domain is SEQ ID NO:1, and the sequence of the β chain variable domain is SEQ ID NO:60.

In another example, the TCR is human-derived.

In another example, the TCR is isolated or purified.

In another example, the TCR is a single chain TCR.

Optionally, the TCR is a single-chain TCR consisting of an α chain variable domain and a β chain variable domain, and the α chain variable domain and the β chain variable domain are connected by a flexible short peptide sequence (linker).

In another example, the TCR comprises an α chain constant region and a β chain constant region, wherein the α chain constant region is a murine constant region and/or the β chain constant region is a murine constant region.

In another example, the TCR comprises an α chain constant region and a β chain constant region, wherein the α chain constant region is a murine constant region and/or the β chain constant region is a murine constant region.

In another example, a conjugate binds to the α chain and/or β chain of the TCR at C- or N-terminal.

Optionally, the conjugate is a detectable label or a therapeutic agent; and further optionally, the therapeutic agent is an anti-CD3 antibody.

In the second aspect of the application, a multivalent TCR complex comprising at least two TCR molecules is provided, and at least one TCR molecule is the TCR of the first aspect.

In the third aspect of the application, a nucleic acid molecule is provided, comprising a nucleotide sequence encoding the TCR molecule of the first aspect of the application or the multivalent TCR complex of the second aspect of the application, or a complement sequence of the nucleotide sequence encoding the TCR molecule of the first aspect of the application or the multivalent TCR complex of the second aspect of the application.

In the fourth aspect of the application, a vector is provided, comprising the nucleic acid molecule of the third aspect of the application.

In the fifth aspect of the application, a host cell is provided, comprising the vector of the fourth aspect of the present application or having the exogenous nucleic acid molecule of the third aspect of the present application integrated into its genome.

In the sixth aspect of the application, an isolated cell is provided, expressing the TCR of the first aspect of the application.

Optionally, the isolated cell is a T cell.

In another example, the cell expresses the TCR of the first aspect of the application and also expresses an exogenous CD8 receptor.

Optionally, the CD8 receptor is CD8a; further optionally, the isolated cell is a T cell.

In the seventh aspect of the application, a pharmaceutical composition is provided, comprising any one or a combination of at least two of the TCR of the first aspect of the application, or the TCR complex of the second aspect of the application, or the isolated cell of the sixth aspect of the application.

In the eighth aspect of the application, a method for treating a disease is provided, comprising administering an appropriate amount of the TCR of the first aspect of the application, or the TCR complex of the second aspect of the application, or the cell of the sixth aspect of the application, or the pharmaceutical composition of the seventh aspect of the application to a subject in need thereof; optionally, the disease is a KRAS G12D-positive tumor, and further optionally, lung cancer, colorectal cancer, pancreatic cancer, gastric cancer, malignant melanoma, human cholangiocarcinoma cells, etc.

In the ninth aspect of the application, a use of the TCR of the first aspect of the application, or the TCR complex of the second aspect of the application, or the isolated cell of the sixth aspect of the application is provided for preparing a specific binding reagent for a target protein.

In another example, the specific binding reagent for a target protein is a diagnostic agent and therapeutic agent for a tumor expressing the target protein; and optionally, the therapeutic agent is directed against a KRAS G12D-positive tumor.

In the tenth aspect of the application, a use of the TCR of the first aspect of the application, or the TCR complex of the second aspect of the application, or the isolated cell of the sixth aspect of the application is provided for preparing a medicament for treating a tumor.

Optionally, the tumor includes a KRAS G12D-positive tumor, and further optionally, lung cancer, colorectal cancer, pancreatic cancer, gastric cancer, malignant melanoma, human cholangiocarcinoma cells, etc.

In the 11^{th} aspect of the application, a method for preparing the TCR of the first aspect of the application is provided, comprising the steps of:
(i) culturing the host cell of the fifth aspect of the application to express the TCR of the first aspect of the application;
(ii) isolating or purifying the TCR.

It is to be understood that within the scope of the application, various technical features of the application and technical features specifically described hereinafter (as in the examples) may be combined with each other to constitute a new or preferred technical solution, which will not be repeated herein one by one due to the limited contents.

The numerical ranges described in the application include not only the point values exemplified above, but also any point values within the above numerical ranges that are not exemplified. The specific point values included in the ranges will not be exhaustively listed, due to limited contents and for the sake of conciseness.

Details of one or more examples of the application are set forth in the following description, and other features, objects, and advantages of the application will become apparent from the specification and claim set thereof.

### DESCRIPTION OF DRAWINGS

FIG. 1 shows the binding curve of the soluble reference TCR, i.e., the wild-type TCR, with the VVVGADGVGK-HLA A1101 complex;

FIG. 2 shows the results of the activation function experiment on the number of IFN-γ spots released by effector cells transfected with the high-affinity TCR of the application against tumor cell lines;

FIG. 3 shows the results of the activation function experiment on the number of granzyme spots released by effector cells transfected with the high-affinity TCR of the application against tumor cell lines;

FIG. 4 shows the results of the LDH experiment on the killing function of effector cells transfected with the high-affinity TCR of the application against tumor cell lines;

FIG. 5 shows the results of the ELISA experiment on the killing function of effector cells transfected with the high-affinity TCR of the application against tumor cell lines;

FIGS. 6a and 6b show the results of the IncuCyte experiment on the killing function of effector cells transfected with the high-affinity TCR of the application against tumor cell lines.

### MODES FOR CARRYING OUT THE INVENTION

Hereafter, the technical solutions in the examples of the application will be clearly and completely described in conjunction with the accompanying drawings in the examples of the application. Obviously, the described examples are only a part of the examples of the application, not all of them. Based on the examples in the application, all other examples obtained by a skilled person without making creative efforts shall fall within the protection scope of the application.

Unless otherwise defined, all technical and scientific terms used herein have the same meanings as commonly understood by a skilled person in the technical field to which the application belongs. The terms used in the specification of the application are only for the purpose of describing specific examples and are not intended to limit the application.

Through extensive and intensive research, a high affinity T cell receptor (TCR) recognizing VVVGADGVGK short peptide (derived from KRAS G12D protein) was obtained, and the VVVGADGVGK short peptide is presented in a form of peptide-HLA A1101 complex. The high affinity TCR has a mutation in three CDR regions of its α chain variable domain:

CDR1α:
SEQ ID NO: 65
TRDTTYY
CDR2α:
SEQ ID NO: 66
RNSFDEQN,
and,
CDR3α:
SEQ ID NO: 67
ALSEAGNDMR;

and/or has a mutation in three CDR regions of its β chain variable domain:

CDR1β:
SEQ ID NO: 116
MNHEY
CDR2β:
SEQ ID NO: 117
SVGEGT
and,
CDR3β:
SEQ ID NO: 118
ASSYLWSYEQY.

Before describing the application, it should be understood that the application is not limited to the described specific methods and experimental conditions, as such methods and conditions may vary. It should also be understood that the terms used herein are only for the purpose of describing specific embodiments and are not intended to be limiting, and the scope of the present application will be limited only by the appended claims.

Unless otherwise defined, all technical and scientific terms used herein have the same meanings as commonly understood by a skilled person in the art to which the application belongs.

Although any methods and materials similar or equivalent to those described in the application can be used in the implementation or testing of the application, preferred methods and materials are exemplified herein.

### Terms

### T Cell Receptor (TCR)

International Immunogenetics Information System (IMGT) can be used to describe a TCR. A native αβ heterodimeric TCR has an α chain and β chain. Generally speaking, each chain comprises a variable region, a junction region and a constant region, and the β chain typically also contains a short hypervariable region between the variable region and junction region, which however is often considered as a part of the junction region. The TCR junction region is determined by the unique TRAJ and TRBJ of IMGT, and the constant region of a TCR is determined by TACT and TRBC of IMGT.

Each variable region comprises three CDRs (complementarity determining regions), CDR1, CDR2 and CDR3, which are chimeric in the framework sequence. In IMGT nomenclature, the different numbers of TRAV and TRBV refer to different Va types and VB types, respectively. In IMGT system, there are following symbols for α chain constant domain: TRAC*01, where “TR” represents T cell receptor gene; “A” represents α chain gene; C represents the constant region; “*01” represents allele gene 1. There are following symbols for β-chain constant domain: TRBC1*01 or TRBC2*01, where “TR” represents T cell receptor gene; “B” represents β-chain gene; C represents constant region; “*01” represents allele gene 1. The constant region of a chain is uniquely defined, and in the form of β chain, there are two possible constant region genes “C1” and “C2”. A skilled person in the art can obtain constant region gene sequences of TCR α and β chains through the disclosed IMGT database.

The α and β chains of TCR are generally considered as having two “domains” respectively, i.e., variable domain and constant domain. The variable domain consists of a connected variable region and a connection region. Therefore, in the specification and claims of the application, “TCR α chain variable domain” refers to a connected TRAV and TRAJ region, and likewise, “TCR β chain variable domain” refers to a connected TRBV and TRBD/TRBJ region. The three CDRs of TCR α chain variable domain are CDR1α, CDR2α and CDR3α, respectively; and the three CDRs of TCR β chain variable domain are CDR1β, CDR2β and CDR3β, respectively. The framework sequences of TCR variable domains of the application may be of murine or human origin, preferably of human origin. The constant domain of TCR comprises an intracellular portion, transmembrane region, and extracellular portion.

In the application, the amino acid sequences of the α and β chain variable domains of the wild type TCR capable of binding to the VVVGADGVGK-HLA A1101 complex are SEQ ID NO: 1 and SEQ ID NO: 2, respectively. In the application, the α chain amino acid sequence and β chain amino acid sequence of the soluble “reference TCR” are SEQ ID NO: 11 and SEQ ID NO: 12, respectively. In the application, the extracellular amino acid sequences of α and β chains of the “wild-type TCR” are SEQ ID NO: 61 and SEQ ID NO: 62, respectively. The TCR sequences used in the application are of human origin. In the application, the α chain and β chain amino acid sequences of the wild type TCR are SEQ ID NO: 63 and SEQ ID NO: 64, respectively. In the application, the terms “polypeptide of the application”, “TCR of the application” and “T cell receptor of the application” can be used interchangeably.

SEQ ID NO: 61
AQKVTQAQTEISVVEKEDVTLDCVYETRDTTYYLFWYKQPPSGEL
VFLIRRNSFDEQNEISGRYSWNFQKSTSSFNFTITASQVVDSAVY
FCALSEAGNDMRFGAGTRLTVKPNIQNPDPAVYQLRDSKSSDKSV
CLFTDFDSQTNVSQSKDSDVYITDKTVLDMRSMDFKSNSAVAWSN
KSDFACANAFNNSIIPEDTFFPSPESS.
SEQ ID NO: 62
NAGVTQTPKFRVLKTGQSMTLLCAQDMNHEYMYWYRQDPGMGLRL
IHYSVGEGTTAKGEVPDGYNVSRLKKQNFLLGLESAAPSQTSVYF
CASSYLWSYEQYFGPGTRLTVTEDLKNVFPPEVAVFEPSEAEISH
TQKATLVCLATGFYPDHVELSWWVNGKEVHSGVSTDPQPLKEQPA
LNDSRYCLSSRLRVSATFWQNPRNHFRCQVQFYGLSENDEWTQDR
AKPVTQIVSAEAWGRAD.
SEQ ID NO: 63
AQKVTQAQTEISVVEKEDVTLDCVYETRDTTYYLFWYKQPPSGEL
VFLIRRNSFDEQNEISGRYSWNFQKSTSSFNFTITASQVVDSAVY
FCALSEAGNDMRFGAGTRLTVKPNIQNPDPAVYQLRDSKSSDKSV
CLFTDFDSQTNVSQSKDSDVYITDKTVLDMRSMDFKSNSAVAWSN
KSDFACANAFNNSIIPEDTFFPSPESSCDVKLVEKSFETDTNLNF
QNLSVIGFRILLLKVAGFNLLMTLRLWSS.
SEQ ID NO: 64
NAGVTQTPKFRVLKTGQSMTLLCAQDMNHEYMYWYRQDPGMGLRL
IHYSVGEGTTAKGEVPDGYNVSRLKKQNFLLGLESAAPSQTSVYF
CASSYLWSYEQYFGPGTRLTVTEDLKNVFPPEVAVFEPSEAEISH
TQKATLVCLATGFYPDHVELSWWVNGKEVHSGVSTDPQPLKEQPA
LNDSRYCLSSRLRVSATFWQNPRNHFRCQVQFYGLSENDEWTQDR
AKPVTQIVSAEAWGRADCGFTSESYQQGVLSATILYEILLGKATL
YAVLVSALVLMAMVKRKDSRG.

### Natural Inter-Chain Disulfide Bond and Artificial Inter-Chain Disulfide Bond

A group of disulfide bonds is present between the Ca and CB chains in the membrane proximal region of a native TCR, which is named herein as “natural inter-chain disulfide bond”. In the application, an inter-chain covalent disulfide bond which is artificially introduced and the position of which is different from the position of a natural inter-chain disulfide bond is named as “artificial inter-chain disulfide bond”.

For convenience of description, in the application, the positions of the amino acid sequences of TRAC*01 and TRBC1*01 or TRBC2*01 are sequentially numbered in order from N-terminal to C-terminal. For example, the 60th amino acid in the order from N-terminal to C-terminal in TRBC1*01 or TRBC2*01 is P (valine), which can be described as Pro60 of TRBC1*01 or TRBC2*01 exon 1 in the application, and can also be expressed as the amino acid at position 60 of TRBC1*01 or TRBC2*01 exon 1. For another example, the 61st amino acid in the order from N-terminal to C-terminal in TRBC1*01 or TRBC2*01 is Q (glutamine), which can be described as Gln61 of TRBC1*01 or TRBC2*01 exon 1 in the application, and can also be expressed as the amino acid at position 61 of TRBC1*01 or TRBC2*01 exon 1, and so on. In the application, the positions of the amino acid sequences of variable regions TRAV and TRBV are numbered according to the positions listed in IMGT. As for an amino acid in TRAV, the position is numbered as 46 in IMGT, which is described in the present invention as the amino acid at position 46 of TRAV, and so on. In the application, if the sequence positions of other amino acids are specifically described, the special description shall prevail.

### Tumor

The term “tumor” refers to include all types of cancer cell growth or carcinogenic processes, metastatic tissues or malignant transformed cells, tissues or organs, regardless of pathological type or stage of infection. Examples of tumors include, without limitation, solid tumors, soft tissue tumors, and metastatic lesions. Examples of solid tumors include: malignant tumors of different organ systems, such as sarcoma, lung squamous cell carcinoma, and cancer. For example: infected prostate, lung, breast, lymph, gastrointestinal (e.g., colon) and genitourinary tract (e.g., kidney, epithelial cells), pharynx.

### DETAILED DESCRIPTION OF THE APPLICATION

It is well known that the α chain variable domain and the β chain variable domain of a TCR contain three CDRs (similar to the complementarity determining regions of antibodies), respectively. CDR3 interacts with the antigen short peptide, and CDR1 and CDR2 interact with HLA. Therefore, the CDRs of a TCR molecule determine its interaction with the antigen short peptide-HLA complex. The amino acid sequences of α chain variable domain and β chain variable domain of a wild type TCR capable of binding the complex of antigen short peptide VVVGADGVGK-HLA A1101 complex (i.e., VVVGADGVGK-HLA A1101 complex) are SEQ ID NO: 1 and SEQ ID NO: 2, respectively. These sequences were firstly discovered by the applicant. It has the following CDR regions:

α chain variable domain CDR:
CDR1α:
SEQ ID NO: 65
TRDTTYY;
CDR2α:
SEQ ID NO: 66
RNSFDEQN;
and,
CDR3α:
SEQ ID NO: 67
ALSEAGNDMR;
and β chain variable domain CDR:
CDR1β:
SEQ ID NO: 116
MNHEY;
CDR2β:
SEQ ID NO: 117
SVGEGT;
and,
CDR3β:
SEQ ID NO: 118
ASSYLWSYEQY.

Moreover, the TCR of the application is an αβ heterodimeric TCR, and the α chain variable domain of the TCR comprises an amino acid sequence having at least 85% (e.g., may be at least 88%, 89%, 90%, 91%, 92%, 93%, 94%, 95%, 96%, 97%, 98%, 99% and the like) sequence homology with the amino acid sequence shown in SEQ ID NO: 1; optionally an amino acid sequence having at least 90% sequence homology; further optionally, an amino acid sequence having at least 92% sequence homology; and further optionally, an amino acid sequence having at least 94% sequence homology; and/or the β chain variable domain of the TCR comprises an amino acid sequence having at least 90% (e.g., may be at least 91%, 92%, 93%, 94%, 95%, 96%, 97%, 98%, 99% sequence homology) sequence homology with the amino acid sequence shown in SEQ ID NO: 2; optionally, an amino acid sequence having at least 92% sequence homology; and further optionally, an amino acid sequence having at least 94% sequence homology.

Moreover, the TCR of the application is a single-chain TCR, and the α chain variable domain of the TCR comprises an amino acid sequence having at least 85% (e.g., may be at least 88%, 89%, 90%, 91%, 92%, 93%, 94%, 95%, 96%, 97%, 98%, 99% and the like) sequence homology with the amino acid sequence shown in SEQ ID NO: 3; optionally an amino acid sequence having at least 90% sequence homology; further optionally, an amino acid sequence having at least 92% sequence homology; and the most preferably, an amino acid sequence having at least 94% sequence homology; the β chain variable domain of the TCR comprises an amino acid sequence having at least 85% (e.g., may be at least 91%, 92%, 93%, 94%, 95%, 96%, 97%, 98%, 99% and the like) sequence homology with the amino acid sequence shown in SEQ ID NO: 4; optionally an amino acid sequence having at least 90% sequence homology; further optionally, an amino acid sequence having at least 92% sequence homology; and the most preferably, an amino acid sequence having at least 94% sequence homology.

In the application, the three CDRs of α chain variable domain SEQ ID NO: 1 of the wild type TCR, i.e., CDR1, CDR2 and CDR3 are located at positions 27-33, 51-58 and 93-102 of SEQ ID NO: 1, respectively. Accordingly, the amino acid residue is numbered as shown in SEQ ID NO: 1, that is, 30T is T at position 4 of CDR1α, 31T is T at position 5 of CDR1α, 32Y is Y at position 6 of CDR1α, 51R is R at position 1 of CDR2a, 52N is N at position 2 of CDR2a, 54F is F at position 4 of CDR2a, 99N is N at position 7 of CDR3α, 100D is D at position 8 of CDR3α, 101M is M at position 9 of CDR3α, and 102R is R at position 10 of CDR3α.

Specific forms of the mutations in the α chain variable domain include one or more groups selected from T30E/Q/D/M/S/V/Y, T31N/D, Y32F, R51Q, N52T/V/Q, F54Y/W, N99R, D100I/M/T/N/Q/L/S, M101T/L/Q/K/H and R102N/H/A/Q/K/S/T/D/V.

In the application, the three CDRs of β chain variable domain SEQ ID NO: 2 of the wild type TCR, i.e., CDR1, CDR2 and CDR3 are located at positions 27-31, 49-54 and 92-102 of SEQ ID NO: 2, respectively. Accordingly, the amino acid residue is numbered as shown in SEQ ID NO: 2, that is, 27M is M at position 1 of CDR1β, 30E is E at position 4 of CDR1β, 51G is G at position 3 of CDR2β, 52E is E at position 4 of CDR2β, 53G is G at position 5 of CDR2β, and 54T is T at position 6 of CDR2β.

Specific forms of the mutations in the β chain variable domain include one or more groups selected from M27N, E30D, G51H, E52Q/N/K/T, G53E/H/D/N/Q/K/R/T, and T54H/S/I.

It should be understood that amino acid names as used herein are identified using internationally accepted single-letter symbols, and their corresponding three-letter abbreviations of amino acid names are as follows: Ala (A), Arg (R), Asn (N), Asp (D), Cys (C), Gln (Q), Glu (E), Gly (G), His (H), Ile (I), Leu (L), Lys (K), Met (M), Phe (F), Pro (P), Ser(S), Thr (T), Trp (W), Tyr (Y), Val (V).

In the application, Pro60 or 60P both refer to proline at position 60. In addition, the expression of the specific form of mutation described in the application, such as “Y50I/M/L”, represents that Y at position 50 is replaced by I, M, or L, and the rest can be deduced by analogy.

Thr48 of the wild type TCR α chain constant region TRAC*01 exon 1 was mutated to cysteine, and Ser57 of the β chain constant region TRBC1*01 or TRBC2*01 exon 1 was mutated to cysteine according to the site-directed mutagenesis method well known to a skilled person in the art, so as to obtain a reference TCR, the amino acid sequences of which are shown in SEQ ID NO: 11 and SEQ ID NO: 12, respectively, and the mutated cysteine residues are indicated by bold letters. The above cysteine substitutions can form an artificial interchain disulfide bond between the constant regions of a and β chain of the reference TCR to form a more stable soluble TCR, so that it is easier to evaluate the binding affinity and/or binding half-life between TCR and VVVGADGVGK-HLA A1101 complex. It will be appreciated that the CDR regions of the TCR variable region determine its affinity for pMHC complex, therefore, the above cysteine substitutions in the TCR constant region won't affect the binding affinity and/or binding half-life of TCR. Therefore, in the present application, the measured binding affinity between the reference TCR and VVVGADGVGK-HLA A1101 complex is considered to be the binding affinity between the wild-type TCR and VVVGADGVGK-HLA A1101 complex. Similarly, if the binding affinity between the TCR of the application and VVVGADGVGK-HLA A1101 complex is determined to be at least 10 times the binding affinity between the reference TCR and VVVGADGVGK-HLA A1101 complex, the binding affinity between the TCR of the application and VVVGADGVGK-HLA A1101 complex is at least 10 times the binding affinity between the wild type TCR and VVVGADGVGK-HLA A1101 complex.

The binding affinity (in inverse proportion to the dissociation equilibrium constant K_{D}) and the binding half-life (expressed as T½) can be determined by any suitable method. It should be understood that doubling of the affinity of the TCR will halve K_{D}. T½ is calculated as In2 divided by dissociation rate (K_{off}). Therefore, doubling of T½ will halve K_{off}. Preferably, the binding affinity or binding half-life of a given TCR is detected for several times by using the same test protocol, for example 3 or more times, and the average of the results is taken. In a preferred embodiment, the affinity of a TCR is detected by the surface plasmon resonance (BIAcore) method in the Examples herein under a condition of 25° C. and pH 7.1-7.5. The dissociation equilibrium constant K_{D} of the reference TCR to VVVGADGVGK-HLA A1101 complex is detected as 1.09E-05M, that is, 10.90 μM by the method, and in the application, the dissociation equilibrium constant K_{D} of the wild type TCR to VVVGADGVGK-HLA A1101 complex is also considered as 10.9 μM. Since doubling of the affinity of TCR will halve K_{D}, if the dissociation equilibrium constant K_{D} of the high affinity TCR to VVVGADGVGK-HLA A1101 complex is detected as 1.09E-06M, i.e., 1.09 UM, the affinity of the high affinity TCR for VVVGADGVGK-HLA A1101 complex is 10 times that of the wild type TCR for VVVGADGVGK-HLA A1101 complex. A skilled person is familiar with the conversion relationship between K_{D} value units, i.e., 1M=10⁶ μM, 1 μM=1000 nM, 1 nM=1000 pM.

Mutations can be carried out by any suitable method including, but not limited to, those based on polymerase chain reaction (PCR), restriction enzyme-based cloning or linkage-independent cloning (LIC) methods. Many standard molecular biology textbooks describe these methods in detail. More details about polymerase chain reaction (PCR) mutagenesis and cloning based on restriction enzymes can be found in Sambrook and Russell, (2001) Molecular Cloning-A Laboratory Manual (Third Edition) CSHL Publishing house. More information about LIC method can be found in Rashtchian, (1995) Curr Opin Biotechnol 6 (1): 30-6.

The method for producing the TCR of the present invention may be, but not limited to, screening for a TCR having high affinity for VVVGADGVGK-HLA A1101 complex from a diverse library of phage particles displaying such TCRs, as described in a literature (Li, et al). (2005) Nature Biotech 23(3): 349-354).

It will be appreciated that genes expressing amino acid of a and β chain variable domain of a wild-type TCR or genes expressing amino acid of α and β chain variable domain of a slightly modified wild-type TCR can be used to prepare template TCRs. Changes necessary to produce the high affinity TCR of the invention are then introduced into the DNA encoding the variable domain of the template TCR.

The high-affinity TCR of the application comprises an α chain variable domain amino acid sequence selected from one of SEQ ID NO: 1 and SEQ ID NO: 13-46; and/or a β chain variable domain amino acid sequence of the TCR selected from one of SEQ ID NO: 2 and SEQ ID NO: 47-60. The amino acid sequences of the α chain variable domain and β chain variable domain of the high-affinity TCR molecule of the application are preferably selected from Table 4 below:

TABLE 4

| | α chain variable | β chain variable |
|---|---|---|
| TCR No. | domain sequence | domain sequence |
| 1 | SEQ ID NO: 13 | SEQ ID NO: 2 |
| 2 | SEQ ID NO: 14 | SEQ ID NO: 2 |
| 3 | SEQ ID NO: 15 | SEQ ID NO: 2 |
| 4 | SEQ ID NO: 16 | SEQ ID NO: 2 |
| 5 | SEQ ID NO: 17 | SEQ ID NO: 2 |
| 6 | SEQ ID NO: 18 | SEQ ID NO: 2 |
| 7 | SEQ ID NO: 19 | SEQ ID NO: 2 |
| 8 | SEQ ID NO: 20 | SEQ ID NO: 2 |
| 9 | SEQ ID NO: 21 | SEQ ID NO: 2 |
| 10 | SEQ ID NO: 22 | SEQ ID NO: 2 |
| 11 | SEQ ID NO: 23 | SEQ ID NO: 2 |
| 12 | SEQ ID NO: 24 | SEQ ID NO: 2 |
| 13 | SEQ ID NO: 25 | SEQ ID NO: 2 |
| 14 | SEQ ID NO: 26 | SEQ ID NO: 2 |
| 15 | SEQ ID NO: 27 | SEQ ID NO: 2 |
| 16 | SEQ ID NO: 28 | SEQ ID NO: 2 |
| 17 | SEQ ID NO: 29 | SEQ ID NO: 2 |
| 18 | SEQ ID NO: 30 | SEQ ID NO: 2 |
| 19 | SEQ ID NO: 31 | SEQ ID NO: 2 |
| 20 | SEQ ID NO: 32 | SEQ ID NO: 2 |
| 21 | SEQ ID NO: 33 | SEQ ID NO: 2 |
| 22 | SEQ ID NO: 34 | SEQ ID NO: 2 |
| 23 | SEQ ID NO: 35 | SEQ ID NO: 2 |
| 24 | SEQ ID NO: 36 | SEQ ID NO: 2 |
| 25 | SEQ ID NO: 37 | SEQ ID NO: 2 |
| 26 | SEQ ID NO: 38 | SEQ ID NO: 2 |
| 27 | SEQ ID NO: 39 | SEQ ID NO: 2 |
| 28 | SEQ ID NO: 40 | SEQ ID NO: 2 |
| 29 | SEQ ID NO: 41 | SEQ ID NO: 2 |
| 30 | SEQ ID NO: 42 | SEQ ID NO: 2 |
| 31 | SEQ ID NO: 43 | SEQ ID NO: 2 |
| 32 | SEQ ID NO: 44 | SEQ ID NO: 2 |
| 33 | SEQ ID NO: 45 | SEQ ID NO: 2 |
| 34 | SEQ ID NO: 46 | SEQ ID NO: 2 |
| 35 | SEQ ID NO: 1 | SEQ ID NO: 47 |
| 36 | SEQ ID NO: 1 | SEQ ID NO: 48 |
| 37 | SEQ ID NO: 1 | SEQ ID NO: 49 |
| 38 | SEQ ID NO: 1 | SEQ ID NO: 50 |
| 39 | SEQ ID NO: 1 | SEQ ID NO: 51 |
| 40 | SEQ ID NO: 1 | SEQ ID NO: 52 |
| 41 | SEQ ID NO: 1 | SEQ ID NO: 53 |
| 42 | SEQ ID NO: 1 | SEQ ID NO: 54 |
| 43 | SEQ ID NO: 1 | SEQ ID NO: 55 |
| 44 | SEQ ID NO: 1 | SEQ ID NO: 56 |
| 45 | SEQ ID NO: 1 | SEQ ID NO: 57 |
| 46 | SEQ ID NO: 1 | SEQ ID NO: 58 |
| 47 | SEQ ID NO: 1 | SEQ ID NO: 59 |
| 48 | SEQ ID NO: 1 | SEQ ID NO: 60 |

Mutated residues in SEQ ID NO: 13-SEQ ID NO: 60 are indicated
in single underline.
SEQ ID NO: 13
AQKVTQAQTEISVVEKEDVTLDCVYETRDTDYYLFWYKQPPSGELVFLIRRNSFDEQNEI
SGRYSWNFQKSTSSFNFTITASQVVDSAVYFCALSEAGNDMRFGAGTRLTVKP.
SEQ ID NO: 14
AQKVTQAQTEISVVEKEDVTLDCVYETRDTNYYLFWYKQPPSGELVFLIRRNSFDEQNEI
SGRYSWNFQKSTSSFNFTITASQVVDSAVYFCALSEAGNDMRFGAGTRLTVKP.
SEQ ID NO: 15
AQKVTQAQTEISVVEKEDVTLDCVYETRDQNYYLFWYKQPPSGELVFLIRRNSFDEQNE
ISGRYSWNFQKSTSSFNFTITASQVVDSAVYFCALSEAGNDMRFGAGTRLTVKP.
SEQ ID NO: 16
AQKVTQAQTEISVVEKEDVTLDCVYETRDENFYLFWYKQPPSGELVFLIRRNSFDEQNEI
SGRYSWNFQKSTSSFNFTITASQVVDSAVYFCALSEAGNDMRFGAGTRLTVKP.
SEQ ID NO: 17
AQKVTQAQTEISVVEKEDVTLDCVYETRDENYYLFWYKQPPSGELVFLIRRNSFDEQNEI
SGRYSWNFQKSTSSFNFTITASQVVDSAVYFCALSEAGNDMRFGAGTRLTVKP.
SEQ ID NO: 18
AQKVTQAQTEISVVEKEDVTLDCVYETRDTTYYLFWYKQPPSGELVFLIRRNSFDEQNEI
SGRYSWNFQKSTSSFNFTITASQVVDSAVYFCALSEAGRITNFGAGTRLTVKP.
SEQ ID NO: 19
AQKVTQAQTEISVVEKEDVTLDCVYETRDTTYYLFWYKQPPSGELVFLIRRNSFDEQNEI
SGRYSWNFQKSTSSFNFTITASQVVDSAVYFCALSEAGRMLHFGAGTRLTVKP.
SEQ ID NO: 20
AQKVTQAQTEISVVEKEDVTLDCVYETRDTTYYLFWYKQPPSGELVFLIRRNSFDEQNEI
SGRYSWNFQKSTSSFNFTITASQVVDSAVYFCALIEAGRTQAFGAGTRLTVKP.
SEQ ID NO: 21
AQKVTQAQTEISVVEKEDVTLDCVYETRDTTYYLFWYKQPPSGELVFLIRRNSFDEQNEI
SGRYSWNFQKSTSSFNFTITASQVVDSAVYFCALSEAGRNTQFGAGTRLTVKP.
SEQ ID NO: 22
AQKVTQAQTEISVVEKEDVTLDCVYETRDTTYYLFWYKQPPSGELVFLIRRNSFDEQNEI
SGRYSWNFQKSTSSFNFTITASQVVDSAVYFCALSEAGRQLKFGAGTRLTVKP.
SEQ ID NO: 23
AQKVTQAQTEISVVEKEDVTLDCVYETRDTTYYLFWYKQPPSGELVFLIRRNSFDEQNEI
SGRYSWNFQKSTSSFNFTITASQVVDSAVYFCALSEAGRTTHFGAGTRLTVKP.
SEQ ID NO: 24
AQKVTQAQTEISVVEKEDVTLDCVYETRDTTYYLFWYKQPPSGELVFLIRRNSFDEQNEI
SGRYSWNFQKSTSSFNFTITASQVVDSAVYFCALSEAGRTKQFGAGTRLTVKP.
SEQ ID NO: 25
AQKVTQAQTEISVVEKEDVTLDCVYETRDTTYYLFWYKQPPSGELVFLIRRNSFDEQNEI
SGRYSWNFQKSTSSFNFTITASQVVDSAVYFCALSEAGRNLAFGAGTRLTVKP.
SEQ ID NO: 26
AQKVTQAQTEISVVEKEDVTLDCVYETRDTTYYLFWYKQPPSGELVFLIRRNSFDEQNEI
SGRYSWNFQKSTSSFNFTITASQVVDSAVYFCALSEAGRNKSFGAGTRLTVKP.
SEQ ID NO: 27
AQKVTQAQTEISVVEKEDVTLDCVYETRDTTYYLFWYKQPPSGELVFLIRRNSFDEQNEI
SGRYSWNFQKSTSSFNFTITASQVVDSAVYFCALSEAGRLTHFGAGTRLTVKP.
SEQ ID NO: 28
AQKVTQAQTEISVVEKEDVTLDCVYETRDTTYYLFWYKQPPSGELVFLIRRNSFDEQNEI
SGRYSWNFQKSTSSFNFTITASQVVDSAVYFCALSEAGPLHSFGAGTRLTVKP.
SEQ ID NO: 29
AQKVTQAQTEISVVEKEDVTLDCVYETRDTTYYLFWYKQPPSGELVFLIRRNSFDEQNEI
SGRYSWNFQKSTSSFNFTITASQVVDSAVYFCALSEAGRQLSFGAGTRLTVKP.
SEQ ID NO: 30
AQKVTQAQTEISVVEKEDVTLDCVYETRDTTYYLFWYKQPPSGELVFLIRRNSFDEQNEI
SGRYSWNFQKSTSSFNFTITASQVVDSAVYFCALSEAGRMKKFGAGTRLTVKP.
SEQ ID NO: 31
AQKVTQAQTEISVVEKEDVTLDCVYETRDTTYYLFWYKQPPSGELVFLIRRNSFDEQNEI
SGRYSWNFQKSTSSFNFTITASQVVDSAVYFCALSEAGRQKTFGAGTRLTVKP.
SEQ ID NO: 32
AQKVTQAQTEISVVEKEDVTLDCVYETRDTTYYLFWYKQPPSGELVFLIRRNSFDEQNEI
SGRYSWNFQKSTSSFNFTITASQVVDSAVYFCALSEAGRLTAFGAGTRLTVKP.
SEQ ID NO: 33
AQKVTQAQTEISVVEKEDVTLDCVYETRDTTYYLFWYKQPPSGELVFLIRRNSFDEQNEI
SGRYSWNFQKSTSSFNFTITASQVVDSAVYFCALSEAGRLLTFGAGTRLTVKP.
SEQ ID NO: 34
AQKVTQAQTEISVVEKEDVTLDCVYETRDTTYYLFWYKQPPSGELVFLIRRNSFDEQNEI
SGRYSWNFQKSTSSFNFTITASQVVDSAVYFCALSEAGRQQTFGAGTRLTVKP.
SEQ ID NO: 35
AQKVTQAQTEISVVEKEDVTLDCVYETRDTTYYLFWYKQPPSGELVFLIRRNSFDEQNEI
SGRYSWNFQKSTSSFNFTITASQVVDSAVYFCALSEAGRSKNFGAGTRLTVKP.
SEQ ID NO: 36
AQKVTQAQTEISVVEKEDVTLDCVYETRDTTYYLFWYKQPPSGELVFLIRRNSFDEQNEI
SGRYSWNFQKSTSSFNFTITASQVVDSAVYFCALSEAGRNTDFGAGTRLTVKP.
SEQ ID NO: 37
AQKVTQAQTEISVVEKEDVTLDCVYETRDTTYYLFWYKQPPSGELVFLIRRNSFDEQNEI
SGRYSWNFQKSTSSFNFTITASQVVDSAVYFCALSEAGRQTVFGAGTRLTVKP.
SEQ ID NO: 38
AQKVTQAQTEISVVEKEDVTLDCVYETRDDNYYLFWYKQPPSGELVFLIRRNSFDEQNE
ISGRYSWNFQKSTSSFNFTITASQVVDSAVYFCALSEAGNDMRFGAGTRLTVKP.
SEQ ID NO: 39
AQKVTQAQTEISVVEKEDVTLDCVYETRDMNYYLFWYKQPPSGELVFLIRRNSFDEQNE
ISGRYSWNFQKSTSSFNFTITASQVVDSAVYFCALSEAGNDMRFGAGTRLTVKP.
SEQ ID NO: 40
AQKVTQAQTEISVVEKEDVTLDCVYETRDSNYYLFWYKQPPSGELVFLIRRNSFDEQNEI
SGRYSWNFQKSTSSFNFTITASQVVDSAVYFCALSEAGNDMRFGAGTRLTVKP
SEQ ID NO: 41
AQKVTQAQTEISVVEKEDVTLDCVYETRDVNYYLFWYKQPPSGELVFLIRRNSFDEQNE
ISGRYSWNFQKSTSSFNFTITASQVVDSAVYFCALSEAGNDMRFGAGTRLTVKP
SEQ ID NO: 42
AQKVTQAQTEISVVEKEDVTLDCVYETRDYNYYLFWYKQPPSGELVFLIRRNSFDEQNE
ISGRYSWNFQKSTSSFNFTITASQVVDSAVYFCALSEAGNDMRFGAGTRLTVKP
SEQ ID NO: 43
AQKVTQAQTEISVVEKEDVTLDCVYETRDTTYYLFWYKQPPSGELVFLIRQTSYDEQNEI
SGRYSWNFQKSTSSFNFTITASQVVDSAVYFCALSEAGNDMRFGAGTRLTVKP
SEQ ID NO: 44
AQKVTQAQTEISVVEKEDVTLDCVYETRDTTYYLFWYKQPPSGELVFLIRQVSFDEQNEI
SGRYSWNFQKSTSSFNFTITASQVVDSAVYFCALSEAGNDMRFGAGTRLTVKP
SEQ ID NO: 45
AQKVTQAQTEISVVEKEDVTLDCVYETRDTTYYLFWYKQPPSGELVFLIRRQSWDEQNE
ISGRYSWNFQKSTSSFNFTITASQVVDSAVYFCALSEAGNDMRFGAGTRLTVKP
SEQ ID NO: 46
AQKVTQAQTEISVVEKEDVTLDCVYETRDTTYYLFWYKQPPSGELVFLIRRTSWDEQNE
ISGRYSWNFQKSTSSFNFTITASQVVDSAVYFCALSEAGNDMRFGAGTRLTVKP
SEQ ID NO: 47
NAGVTQTPKFRVLKTGQSMTLLCAQDMNHEYMYWYRQDPGMGLRLIHYSVHEGTTA
KGEVPDGYNVSRLKKQNFLLGLESAAPSQTSVYFCASSYLWSYEQYFGPGTRLTVT
SEQ ID NO: 48
NAGVTQTPKFRVLKTGQSMTLLCAQDMNHEYMYWYRQDPGMGLRLIHYSVGEETTAK
GEVPDGYNVSRLKKQNFLLGLESAAPSQTSVYFCASSYLWSYEQYFGPGTRLTVT
SEQ ID NO: 49
NAGVTQTPKFRVLKTGQSMTLLCAQDMNHEYMYWYRQDPGMGLRLIHYSVGEHTTA
KGEVPDGYNVSRLKKQNFLLGLESAAPSQTSVYFCASSYLWSYEQYFGPGTRLTVT
SEQ ID NO: 50
NAGVTQTPKFRVLKTGQSMTLLCAQDMNHEYMYWYRQDPGMGLRLIHYSVGEGHTA
KGEVPDGYNVSRLKKQNFLLGLESAAPSQTSVYFCASSYLWSYEQYFGPGTRLTVT
SEQ ID NO: 51
NAGVTQTPKFRVLKTGQSMTLLCAQDMNHEYMYWYRQDPGMGLRLIHYSVGEDTTA
KGEVPDGYNVSRLKKQNFLLGLESAAPSQTSVYFCASSYLWSYEQYFGPGTRLTVT
SEQ ID NO: 52
NAGVTQTPKFRVLKTGQSMTLLCAQDMNHEYMYWYRQDPGMGLRLIHYSVGENTTA
KGEVPDGYNVSRLKKQNFLLGLESAAPSQTSVYFCASSYLWSYEQYFGPGTRLTVT
SEQ ID NO: 53
NAGVTQTPKFRVLKTGQSMTLLCAQDMNHEYMYWYRQDPGMGLRLIHYSVGEQTTA
KGEVPDGYNVSRLKKQNFLLGLESAAPSQTSVYFCASSYLWSYEQYFGPGTRLTVT
SEQ ID NO: 54
NAGVTQTPKFRVLKTGQSMTLLCAQDMNHEYMYWYRQDPGMGLRLIHYSVGEKTTA
KGEVPDGYNVSRLKKQNFLLGLESAAPSQTSVYFCASSYLWSYEQYFGPGTRLTVT
SEQ ID NO: 55
NAGVTQTPKFRVLKTGQSMTLLCAQDMNHEYMYWYRQDPGMGLRLIHYSVGERTTAK
GEVPDGYNVSRLKKQNFLLGLESAAPSQTSVYFCASSYLWSYEQYFGPGTRLTVT
SEQ ID NO: 56
NAGVTQTPKFRVLKTGQSMTLLCAQDMNHEYMYWYRQDPGMGLRLIHYSVGQHSTA
KGEVPDGYNVSRLKKQNFLLGLESAAPSQTSVYFCASSYLWSYEQYFGPGTRLTVT
SEQ ID NO: 57
NAGVTQTPKFRVLKTGQSMTLLCAQDMNHEYMYWYRQDPGMGLRLIHYSVGNTITAK
GEVPDGYNVSRLKKQNFLLGLESAAPSQTSVYFCASSYLWSYEQYFGPGTRLTVT
SEQ ID NO: 58
NAGVTQTPKFRVLKTGQSMTLLCAQDMNHEYMYWYRQDPGMGLRLIHYSVGKEITAK
GEVPDGYNVSRLKKQNFLLGLESAAPSQTSVYFCASSYLWSYEQYFGPGTRLTVT
SEQ ID NO: 59
NAGVTQTPKFRVLKTGQSMTLLCAQDMNHEYMYWYRQDPGMGLRLIHYSVGTTTTAK
GEVPDGYNVSRLKKQNFLLGLESAAPSQTSVYFCASSYLWSYEQYFGPGTRLTVT
SEQ ID NO: 60
NAGVTQTPKFRVLKTGQSMTLLCAQDNNHDYMYWYRQDPGMGLRLIHYSVGEGTTAK
GEVPDGYNVSRLKKQNFLLGLESAAPSQTSVYFCASSYLWSYEQYFGPGTRLTVT

For the purposes of the application, the TCR of the application is a moiety having at least one TCR α and/or TCR β chain variable domain. They usually comprise both of TCR α chain variable domain and TCR β chain variable domain. They may be αβ heterodimers or single-chain forms or any other stable forms. In adoptive immunotherapy, the full length chain of the αβ heterodimeric TCR (including the cytoplasmic and transmembrane domains) can be transfected. The TCR of the application can be used as a targeting agent for delivering a therapeutic agent to an antigen presenting cell or in combination with other molecules to prepare a bifunctional polypeptide to direct effector cells, when the TCR is preferably in a soluble form.

For stability, it is disclosed in the prior art that a soluble and stable TCR molecule can be obtained by introducing an artificial interchain disulfide bond between the α and β chain constant domains of a TCR, as described in PCT/CN2015/093806. Therefore, the TCR of the application may be a TCR that an artificial interchain disulfide bond is introduced between the residues of its α and β chain constant domains. Cysteine residues form an artificial interchain disulfide bond between the α and β chain constant domains of the TCR. A cysteine residue can replace other amino acid residue at a suitable position in a native TCR to form an artificial interchain disulfide bond. For example, Thr48 of TRAC*01 exon 1 and Ser57 of TRBC1*01 or TRBC2*01 exon 1 can be replaced to form a disulfide bond. Other sites for introducing a cysteine residue to form a disulfide bond may be:
Thr45 of TRAC*01 exon 1 and Ser77 of TRBC1*01 or TRBC2*01 exon 1;
Tyr10 of TRAC*01 exon 1 and Ser17 of TRBC1*01 or TRBC2*01 exon 1;
Thr45 of TRAC*01 exon 1 and Asp59 of TRBC1*01 or TRBC2*01 exon 1;
Ser15 of TRAC*01 exon 1 and Glu15 of TRBC1*01 or TRBC2*01 exon 1;
Arg53 of TRAC*01 exon 1 and Ser54 of TRBC1*01 or TRBC2*01 exon 1;
Pro89 of TRAC*01 exon 1 and Ala19 of TRBC1*01 or TRBC2*01 exon 1;
or Tyr10 of TRAC*01 exon 1 and Glu20 of TRBC1*01 or TRBC2*01 exon 1.

That is, cysteine residues replace any group of the above-mentioned sites in a and β chain constant domains. A maximum of 15, or a maximum of 10, or a maximum of 8 or fewer amino acids may be truncated at one or more C-termini of the constant domain of the TCR of the application such that it does not include cysteine residues to achieve the purpose of deleting native interchain disulfide bonds, or the cysteine residues forming a natural interchain disulfide bond can also be mutated to another amino acid for achieving the above purpose.

As described above, the TCR of the present invention may comprise an artificial interchain disulfide bond introduced between residues of its α and β chain constant domains. It should be noted that the introduced artificial disulfide bond as described above can be contained or not contained between the constant domains, and the TCR of the present invention may contain a TRAC constant domain sequence and a TRBC1 or TRBC2 constant domain sequence. The TRAC constant domain sequence and the TRBC1 or TRBC2 constant domain sequence of the TCR can be joined by a natural interchain disulfide bond present in the TCR.

Additionally, as for stability, it was also disclosed in a patent literature PCT/CN2016/077680 that the introduction of an artificial interchain disulfide bond between a chain variable region and β chain constant region of a TCR can significantly improve the stability of the TCR. Therefore, an artificial interchain disulfide bond may be contained between α chain variable region and β chain constant region of a high affinity TCR of the application. Cysteine residues forming an artificial interchain disulfide bond between α chain variable region and β chain constant region of the TCR is substituted for:
an amino acid at position 46 of TRAV and amino acid at position 60 of TRBC1*01 or TRBC2*01 exon 1;
an amino acid at position 47 of TRAV and amino acid at position 61 of TRBC1*01 or TRBC2*01 exon 1;
an amino acid at position 46 of TRAV and amino acid at position 61 of TRBC1*01 or TRBC2*01 exon 1;
or an amino acid at position 47 of TRAV and amino acid at position 60 of TRBC1*01 or TRBC2*01 exon 1.

Optionally, such a TCR may comprises (i) all or part of TCR α chain other than its transmembrane domain, and (ii) all or part of TCR β chain other than its transmembrane domain, wherein both of (i) and (ii) comprise the variable domain and at least a portion of constant domains of the TCR chain, and the α chain and β chain form a heterodimer.

Further optionally, such TCR may comprise α chain variable domain and β chain variable domain and all or part of β chain constant domain other than the transmembrane domain, which, however, does not comprise α chain constant domain, and the α chain variable domain of the TCR and the β chain form a heterodimer.

For stability, in another aspect, the TCR of the application also includes a TCR having a mutation in its hydrophobic core region, and these mutations in hydrophobic core region are preferably mutations capable of increasing the stability of the TCR of the application, as described in WO 2014/206304. Such a TCR can have mutations at following positions in the variable domain hydrophobic core: (a and/or β chain) variable region amino acids at position 11, 13, 19, 21, 53, 76, 89, 91, 94, and/or α chain J gene (TRAJ) short peptide amino acid at reciprocal positions 3, 5, 7 and/or β chain J gene (TRBJ) short peptide amino acid at reciprocal positions 2, 4, 6, wherein the positions in amino acid sequence are numbered according to the position numbers listed in the International Immunogenetics Information System (IMGT). A skilled person in the art will know the above-described international immunogenetic information system and can obtain the position numbers of the amino acid residues of different TCRs in the IMGT based on the database.

In the application, a TCR in which there is a mutation in the hydrophobic core region may be a high-stability single-chain TCR consisting of TCR α and β chain variable domains that linked by a flexible peptide chain. The CDR regions of TCR variable region determine its affinity for the short peptide-HLA complex, and mutations in hydrophobic core can increase the stability of the TCR, but won't affect its affinity for the short peptide-HLA complex. It should be noted that the flexible peptide chain in the application may be any peptide chain suitable for linking TCR α and β chain variable domains. The template chain constructed in Example 1 of the application for screening high-affinity TCRs is a high-stability single-chain TCR containing mutations in hydrophobic core as described above. The affinity between a TCR and VVVGADGVGK-HLA A1101 complex can be easily evaluated by using a TCR with higher stability.

The CDR regions of α chain variable domain and β chain variable domain of the single chain template TCR are identical to the CDR regions of the wild type TCR. That is, the three CDRs of α chain variable domain are CDR1α: TRDTTYY, CDR2α: RNSFDEQN, CDR3α: ALSEAGNDMR and the three CDRs of β chain variable domains are CDR1β: MNHEY, CDR2β: SVGEGT, CDR3β: ASSYLWSYEQY, respectively. The amino acid sequence of the single-chain template TCR is shown in SEQ ID NO: 9, and the nucleotide sequence of the single-chain template TCR is shown in SEQ ID NO: 10, respectively, thereby screening a single-chain TCR consisting of α chain variable domain and β chain variable domain and having high affinity for VVVGADGVGK-HLA A1101 complex.

The αβ heterodimer of the application having high affinity for VVVGADGVGK-HLA A1101 complex was obtained by transferring the CDR regions of α and β chain variable domains of the selected high affinity single-chain TCR to the corresponding positions of α chain variable domain (SEQ ID NO: 1) and β chain variable domain (SEQ ID NO: 2) of a wild type TCR.

The TCR of the application can be provided in a form of multivalent complex. The multivalent TCR complex of the application comprises a polymer formed by combining two, three, four or more TCRs of the present invention, for example, a tetrameric domain of p53 can be used to produce a tetramer. Alternatively, more TCRs of the application can be combined with another molecule to form a complex. The TCR complexes of the application can be used to track or target cells that present a particular antigen in vitro or in vivo, or produce intermediates of other multivalent TCR complexes with such uses.

The TCR of the application may be used alone or combined with a conjugate in a covalent manner or other manner, preferably in a covalent manner. The conjugate includes a detectable label (for diagnostic purposes, wherein the TCR is used to detect the presence of a cell presenting VVVGADGVGK-HLA A1101 complex), a therapeutic agent, a PK (protein kinase) modifying moiety, or combination of any of the substances as described above.

Detectable labels for diagnostic purposes include, but are not limited to, fluorescent or luminescent labels, radioactive labels, MRI (magnetic resonance imaging) or CT (electron computed tomography) contrast agents, or enzymes capable of producing detectable products.

Therapeutic agents that can be combined with or coupled to the TCRs of the application include, but are not limited to:
1. Radionuclides (Koppe et al., 2005, Cancer metastasis reviews 24, 539);
2. Biotoxin (Chaudhary et al., 1989, Nature 339, 394; Epel et al., 2002, Cancer Immunology and Immunotherapy 51, 565);
3. Cytokines, such as IL-2, etc. (Gillies et al., 1992, National Academy of Sciences (PNAS) 89, 1428; Card et al., 2004, Cancer Immunology and Immunotherapy 53, 345; Halin et al., 2003, Cancer Research 63, 3202);
4. Antibody Fc fragment (Mosquera et al., 2005, The Journal Of Immunology 174, 4381);
5. Antibody scFv fragments (Zhu et al., 1995, International Journal of Cancer 62, 319);
6. Gold nanoparticles/Nanorods (Lapotko et al., 2005, Cancer letters 239, 36; Huang et al., 2006, Journal of the American Chemical Society 128, 2115);
7. Viral particles (Peng et al., 2004, Gene therapy 11, 1234);
8. Liposomes (Mamot et al., 2005, Cancer research 65, 11631);
9. Nanomagnetic particles;
10. Prodrug activating enzymes (e.g., DT-diaphorase (DTD) or biphenyl hydrolase-like protein (BPHL);
11. Chemotherapeutic agent (e.g., cisplatin) or any form of nanoparticles, and the like.

An antibody to which the TCR of the application binds or a fragment thereof includes an anti-T cell or an NK-cell determining antibody, such as an anti-CD3 or anti-CD28 or anti-CD16 antibody, and the above antibody or a fragment thereof binds to a TCR, thereby better directing effector cells to target cells. In a preferred embodiment, the TCR of the application binds to an anti-CD3 antibody or a functional fragment or variant thereof. A fusion molecule of the TCR of the application and an anti-CD3 single-chain antibody comprises a TCR α chain variable domain, the amino acid sequence of which is selected from the group consisting of SEQ ID NO: 1, and SEQ ID NO: 13-46, and/or a TCR β chain variable domain, the amino acid sequence of which is selected from the group consisting of SEQ ID NO: 2, and SEQ ID NO: 47-60.

The application also relates to a nucleic acid molecule encoding the TCR of the application. The nucleic acid molecule of the application may be in a form of DNA or RNA. DNA can be a coding strand or a non-coding strand. For example, a nucleic acid sequence encoding the TCR of the application may be the same as the nucleic acid sequence shown in the application or a degenerate variant thereof. By way of example, “degenerate variant”, as used herein, refers to a nucleic acid sequence which encodes a protein with a sequence of SEQ ID NO: 3, but is differences from the sequence of SEQ ID NO: 5.

The full length sequence of the nucleic acid molecule of the application or a fragment thereof can generally be obtained by, but not limited to, PCR amplification, recombinant methods or synthetic methods. At present, it is possible to obtain a DNA sequence encoding the TCR (or a fragment thereof, or a derivative thereof) of the application completely by chemical synthesis. And then the DNA sequence can be introduced into various existing DNA molecules (or vectors) and cells known in the art.

The application also relates to a vector comprising the nucleic acid molecule of the application, as well as a host cell genetically engineered using the vector or coding sequence of the application.

The application also encompasses isolated cells, particularly T cells, which express the TCR of the application. There are a number of methods suitable for T cell transfection with DNA or RNA encoding the high affinity TCR of the application (e.g., Robbins et al., (2008) J. Immunol. 180:6116-6131). T cells expressing the high affinity TCR of the application can be used in adoptive immunotherapy. A skilled person in the art can know many suitable methods for performing adoptive therapy (e.g., Rosenberg et al., (2008) Nat Rev Cancer 8 (4): 299-308).

The application also provides a pharmaceutical composition, comprising a pharmaceutically acceptable carrier and a TCR of the application, or a TCR complex of the application, or cells presenting the TCR of the application.

The invention also provides a method for treating a disease, comprising administering to a subject in need thereof an appropriate amount of a TCR of the application, or a TCR complex of the application, or cells presenting a TCR of the application, or a pharmaceutical composition of the application.

In the art, when an amino acid with similar properties is used for substitution, the function of the protein is usually not altered. The addition of one or several amino acids at C-terminus and/or N-terminus generally does not alter the structure and function of the protein. Therefore, the TCR of the application further includes a TCR, wherein up to 5, preferably up to 3, more preferably up to 2, the most preferably 1 amino acid (especially an amino acid located outside CDR regions) of the TCR of the application is replaced by an amino acid with similar properties and still be able to maintain its function.

The present application also includes a TCR obtained from the TCR of the present application by slight modification. Form of modification (usually without altering the primary structure) includes: chemically derived forms of the TCR of the application, such as acetylation or carboxylation. Modifications also include glycosylation, such as those TCRs produced by glycosylation modifications in the synthesis and processing or in further processing steps of the TCR of the application. Such modification can be accomplished by exposing the TCR to an enzyme performing glycosylation (such as a mammalian glycosylation enzyme or a deglycosylation enzyme). Modification forms also include sequences having phosphorylated amino acid residues (such as phosphotyrosine, phosphoserine, phosphothreonine). Also included are TCRs that have been modified to enhance their antiproteolytic properties or optimize solubility properties.

The TCR, TCR complexes of the application or T cells transfected by the TCRs of the application can be provided in a pharmaceutical composition together with a pharmaceutically acceptable carrier. The TCR, multivalent TCR complex or cell of the application is typically provided as part of a sterile pharmaceutical composition, which typically comprises a pharmaceutically acceptable carrier. The pharmaceutical composition can be of any suitable form (depending on the desired method for administration to a patient). It can be provided in a unit dosage form, usually in a sealed container, and can be provided as part of a kit. Such kit includes (but not necessary) instructions. It can include a plurality of said unit dosage form.

Furthermore, the TCR of the application may be used alone or in combination with other therapeutic agents (e.g., formulated in the same pharmaceutical composition).

The pharmaceutical composition may also contain a pharmaceutically acceptable carrier. The term “pharmaceutically acceptable carrier” refers to a carrier for the administration of a therapeutic agent. The term refers to such pharmaceutical carriers which themselves do not induce the production of antibodies harmful to the individual receiving the composition and which are not excessively toxic after administration. These carriers are well known to a skilled person in the art. A full discussion of pharmaceutically acceptable excipients can be found in Remington's Pharmaceutical Sciences (Mack Pub. Co., N. J. 1991). Such carriers include, but are not limited to, saline, buffer, dextrose, water, glycerol, ethanol, adjuvants, and combinations thereof.

The pharmaceutically acceptable carrier in the therapeutic composition may contain a liquid such as water, saline, glycerol and ethanol. In addition, auxiliary substances such as wetting or emulsifying agents, pH buffering substances and the like may also be present in these carriers.

In general, the therapeutic compositions can be formulated as injectables, such as liquid solutions or suspensions; and solid forms such as liquid carriers, which may be suitable for being formulated in solution or suspension prior to injection.

Once a composition of the application is formulated, it can be administered by conventional routes including, but not limited to, intraocular, intramuscular, intravenous, subcutaneous, intradermal, or topical administration, preferably parenteral, including subcutaneous, intramuscular or intravenous administration. A subject to be prevented or treated may be an animal; especially a human.

When the pharmaceutical composition of the application is used for actual treatment, pharmaceutical compositions of various dosage forms may be employed depending on the uses, preferably, an injection, an oral preparation, or the like.

These pharmaceutical compositions can be formulated by mixing, diluting or dissolving according to conventional methods, occasionally, suitable pharmaceutical additives can be added such as excipients, disintegrating agents, binders, lubricants, diluents, buffers, isotonicity Isotonicities, preservatives, wetting agents, emulsifiers, dispersing agents, stabilizers and co-solvents, and the formulation process can be carried out in a customary manner depending on the dosage form.

The pharmaceutical composition of the application can also be administered in the form of a sustained release preparation. For example, the TCR of the application can be incorporated into a pill or microcapsule in which the sustained release polymer is used as a carrier, and then the pill or microcapsule is surgically implanted into the tissue to be treated. Examples of the sustained-release polymer include ethylene-vinyl acetate copolymer, polyhydrometaacrylate, polyacrylamide, polyvinylpyrrolidone, methylcellulose, lactic acid polymer, lactic acid-glycolic acid copolymer or the like, preferably biodegradable polymer, such as lactic acid polymer and lactic acid-glycolic acid copolymer.

When the pharmaceutical composition of the application is used for actual treatment, the amount of the TCR or TCR complex of the application or the cell presenting the TCR of the application as an active ingredient may be reasonably determined based on the body weight, age, sex, and degree of symptoms of each patient to be treated, and ultimately by a doctor.

### Main Advantages of the Application

(1) The high-affinity TCR of the application can specifically bind to the VVVGADGVGK-HLA A1101, and the cells transfected with the high-affinity TCR of the application can be specifically activated;
(2) The effector cells transfected with the high-affinity TCR of the application have a strong specific killing effect.

The application is further illustrated by the following specific examples. It is to be understood that these examples are for illustrative purposes only and are not intended to limit the scope of the application. The experimental methods in the following examples which do not specify the specific conditions are usually performed under conventional conditions, for example, conditions described in Sambrook and Russell et al., Molecular Cloning-A Laboratory Manual (Third Edition) (2001) CSHL Publishing company, or in accordance with the conditions recommended by the manufacturer. Percentages and parts are by weight unless otherwise stated.

### Materials and Method

The experimental materials used in the examples of the application can commercially available, unless otherwise specified, among which E. coli DH5a was purchased from Tiangen, E. coli BL21 (DE3) was purchased from Tiangen, E. coli Tuner (DE3) was purchased from Novagen, and plasmid pET28a was purchased from Novagen.

### Example 1. Generation of Stable Single-Chained TCR Template Chains with Mutations in Hydrophobic Core

In the application, a method of site-directed mutagenesis was used according to a patent literature WO2014/206304 to construct a stable single-chain TCR molecule consisting of TCR α and β chain variable domain connected by a flexible short peptide, and the amino acid and DNA sequences of the single-chain TCR molecule are SEQ ID NO: 9 and SEQ ID NO: 10, respectively. The single-chain TCR molecule was used as a template for screening high-affinity TCR molecules. The amino acid sequences of α variable domain of the template chain is SEQ ID NO: 3 and the amino acid sequences of β variable domain of the template chain is SEQ ID NO: 4; the corresponding nucleotide sequences are SEQ ID NO: 5 and 6, respectively; and the amino acid sequence and nucleotide sequence of the flexible short linker are SEQ ID NO: 7 and 8, respectively.

The target gene carrying the template chain was digested with NcoI and NotI, and ligated with pET28a vector digested with NcoI and NotI. The ligation product was transformed into E. coli DH5α, plated on a kanamycin-containing LB plate, inverted and cultured at 37° C. overnight, and the positive clones were picked for PCR screening. Positive recombinants were sequenced to determine the correct sequence and the recombinant plasmid was extracted and transferred into E. coli BL21 (DE3) for expression.

SEQ ID NO: 3
AQKVTQSQTELSVVEGEDVTIDCVYETRDTTYYLFWYKQPPSGEPVFLIR
RNSFDEQNEISGRYSWNFQKSTSSFNFTITDVQPNDSAVYFCALSEAGND
MRFGAGTRLTVKP.
SEQ ID NO: 4
NAGVTQTPKYLSVKTGQSVTLQCAQDMNHEYMYWYRQDPGQGLRLIYYSV
GEGTTAKGEVPDRYNVSRLKKQNFLLGIESVTPSDTSVYFCASSYLWSYE
QYFGPGTRLTVT.
SEQ ID NO: 5
Gctcaaaaagttactcaaagccaaaccgagctgagcgtggttgagggtga
agacgtgaccatcgattgcgtttatgaaacccgtgacaccacctactacc
tgttctggtacaagcaaccgccgagcggcgagccggttttcctgatccgt
cgtaacagctttgatgagcagaacgaaattagcggccgttatagctggaa
ctttcagaagagcaccagcagcttcaactttaccattaccgacgtgcagc
cgaacgatagcgcggtttacttctgcgcgctgagcgaagcgggtaacgac
atgcgttttggtgcgggtacccgtctgaccgtgaaaccg.
SEQ ID NO: 6
Aacgcgggcgttacccagaccccgaagtatctgagcgtgaaaaccggtca
aagcgttaccctgcagtgcgcgcaagacatgaaccacgagtacatgtatt
ggtaccgtcaggacccgggtcaaggcctgcgtctgatctactatagcgtg
ggcgagggcaccaccgcgaaaggtgaagtgccggaccgttacaacgttag
ccgtctgaagaaacagaacttcctgctgggcattgagagcgtgaccccga
gcgataccagcgtttatttctgcgcgagcagctacctgtggagctatgaa
caatactttggtccgggcacccgtctgaccgttacc.
SEQ ID NO: 7
GGGSEGGGSEGGGSEGGGSEGGTG.
SEQ ID NO: 8
ggtggcggtagcgagggcgctggcagcgaaggtggcggtagcgagggcgg
tggcagcgaaggtggcaccggt.
SEQ ID NO: 9
AQKVTQSQTELSVVEGEDVTIDCVYETRDTTYYLFWYKQPPSGEPVFLIR
RNSFDEQNEISGRYSWNFQKSTSSFNFTITDVQPNDSAVYFCALSEAGND
MRFGAGTRLTVKPGGGSEGGGSEGGGSEGGGSEGGTGNAGVTQTPKYLSV
KTGQSVTLQCAQDMNHEYMYWYRQDPGQGLRLIYYSVGEGTTAKGEVPDR
YNVSRLKKQNFLLGIESVTPSDTSVYFCASSYLWSYEQYFGPGTRLTVT.
SEQ ID NO: 10
Gctcaaaaagttactcaaagccaaaccgagctgagcgtggttgagggtga
agacgtgaccatcgattgcgtttatgaaacccgtgacaccacctactacc
tgttctggtacaagcaaccgccgagcggcgagccggttttcctgatccgt
cgtaacagctttgatgagcagaacgaaattagcggccgttatagctggaa
ctttcagaagagcaccagcagcttcaactttaccattaccgacgtgcagc
cgaacgatagcgcggtttacttctgcgcgctgagcgaagcgggtaacgac
atgcgttttggtgcgggtacccgtctgaccgtgaaaccgggtggcggtag
cgagggcggtggcagcgaaggtggcggtagcgagggcggtggcagcgaag
gtggcaccggtaacgcgggcgttacccagaccccgaagtatctgagcgtg
aaaaccggtcaaagcgttaccctgcagtgcgcgcaagacatgaaccacga
gtacatgtattggtaccgtcaggacccgggtcaaggcctgcgtctgatct
actatagcgtgggcgagggcaccaccgcgaaaggtgaagtgccggaccgt
tacaacgttagccgtctgaagaaacagaacttcctgctgggcattgagag
cgtgaccccgagcgataccagcgtttatttctgcgcgagcagctacctgt
ggagctatgaacaatactttggtccgggcacccgtctgaccgttacc.

### Example 2. Expression, Renaturation and Purification of the Stable Single-Chain TCR Constructed in Example 1

All of BL21 (DE 3) colonies containing the recombinant plasmid pET28a-template chain prepared in Example 1 were inoculated into LB medium containing kanamycin, and cultured at 37° C. until OD600 was 0.6-0.8. IPTG was added to a final concentration of 0.5 mM, and cultured at 37° C. for another 4 hrs. The cell pellets were harvested by centrifugation at 5000 rpm for 15 mins, and the cell pellets were lysed with Bugbuster Master Mix (Merck). The inclusion bodies were recovered by centrifugation at 6000 rpm for 15 min, followed by washing with Bugbuster (Merck) to remove cell debris and membrane fraction. The inclusion bodies were collected by centrifugation at 6000 rpm for 15 min, and dissolved in a buffer (20 mM Tris-HCl pH 8.0, 8 M urea), and the insoluble matters were removed by high-speed centrifugation. The supernatant was quantitatively determined by BCA method, and then dispensed and stored at −80° C. until use.

To 5 mg of dissolved single-chain TCR inclusion body protein, 2.5 mL of buffer (6 M Gua-HCl, 50 mM Tris-HCl pH 8.1, 100 mM NaCl, 10 mM EDTA) was added, then DTT was added to a final concentration of 10 mM, and incubated at 37° C. for 30 min. The single-chain TCRs as treated above was added dropwise to a 125 mL of refolding buffer (100 mM Tris-HCl pH 8.1, 0.4 M L-arginine, 5 M urea, 2 mM EDTA, 6.5 mM β-mercapthoethylamine, 1.87 mM Cystamine) with a syringe, and stirred at 4° C. for 10 min. Then the refolded solution was loaded into a cellulose membrane dialysis bag with a cut-off of 4 kDa, and the dialysis bag was placed in 1 L of pre-cooled water, and stirred slowly at 4° C. overnight. After 17 hours, the dialysis liquid was changed to 1 L of pre-chilled buffer (20 mM Tris-HCl pH 8.0) and dialysis was continued for 8 h at 4° C. The dialysis liquid was then replaced with the same fresh buffer and dialysis was continued overnight. After 17 hours, the sample was filtered through a 0.45 μm filter, vacuum degassed and purified through an anion exchange column (HiTrap Q HP, GE Healthcare) with a linear gradient elution of 0-1 M NaCl prepared with 20 mM Tris-HCl pH 8.0. The collected fractions were subjected to SDS-PAGE analysis, and the fractions containing single-chain TCRs were concentrated and further purified by a gel filtration column (Superdex 75 10/300, GE Healthcare), and the target components were also subjected to SDS-PAGE analysis.

The eluted fractions for BIAcore analysis was further tested for purity using gel filtration. The conditions were as follows: chromatographic column Agilent Bio SEC-3 (300 A, φ 7.8×300 mm), mobile phase 150 mM phosphate buffer, flow rate 0.5 mL/min, column temperature 25° C., and UV detection wavelength 214 nm.

### Example 3. Binding Characterization

### BIAcore Analysis

The binding activity of the TCR molecule to VVVGADGVGK-HLA A1101 complex was detected using BIAcore T200 real-time analysis system. The anti-streptavidin antibody (GenScript) was added to a coupling buffer (10 mM sodium acetate buffer, pH 4.77), and then the antibody was passed through a CM5 chip pre-activated with EDC and NHS to immobilize the antibody on the surface of the chip. The unreacted activated surface was finally blocked with a solution of ethanolamine in hydrochloric acid to complete the coupling process at a coupling level of about 15,000 RU.

A low concentration of streptavidin flowed over the surface of the antibody-coated chip, then VVVGADGVGK-HLA A1101 complex flowed through the detection channel with another channel being used as a reference channel. 0.05 mM biotin flowed over the chip for 2 min at a flow rate of 10 μL/min, thereby blocking the remaining binding sites for streptavidin. The affinity was determined by single-cycle kinetic analysis. TCR was diluted to several different concentrations with HEPES-EP buffer (10 mM HEPES, 150 mM NaCl, 3 mM EDTA, 0.005% P20, pH 7.4), and flowed over the surface of the chip in turn at a flow rate of 30 μL/min, with a binding time of 120 s per injection. After the last injection, the chip was placed for dissociation for 600 s. At the end of each round of assay, the chip was regenerated with 10 mM Gly-HCl, pH 1.75. Kinetic parameters were calculated using BIAcore Evaluation software.

The preparation process for the above VVVGADGVGK-HLA A1101 complex is described as follows:
a. Purification: 100 ml of E. coli liquid induced to express heavy or light chain was collected, and centrifuged at 8000 g for 10 min at 4° C., and the cells were washed once with 10 ml of PBS, and then vigorously shaken in 5 ml of BugBuster Master Mix Extraction Reagents (Merck) for resuspending the cells. The suspension was incubated for 20 min at room temperature, and then centrifuged at 6000 g for 15 min at 4° C. The supernatant was discarded to collect inclusion bodies.

The above inclusion bodies was resuspended in 5 ml BugBuster Master Mix and incubated vortically at room temperature for 5 min. 30 ml of 10 time-diluted BugBuster was added, mixed, and centrifuged at 6000 g for 15 min at 4° C. The supernatant was discarded, 30 ml of 10 time-diluted BugBuster was added to resuspend the inclusion body, mixed, and centrifuged twice at 6000 g at 4° C. for 15 min. 30 ml of 20 mM Tris-HCl pH 8.0 was added to resuspend the inclusion bodies, mixed, and centrifuged at 6000 g at 4° C. for 15 min. Finally, inclusion bodies were dissolved in 20 mM Tris-HCl 8M urea, and the purity of inclusion bodies was determined by SDS-PAGE and the concentration was measured by BCA kit.
b. Refolding: Synthesized short peptide VVVGADGVGK (Synthesized by Jiangsu GenScript Biotech Co., Ltd.) were dissolved in DMSO to a concentration of 20 mg/ml. Inclusion bodies of light and heavy chains were solubilized in a solution comprising 8 M urea, 20 mM Tris pH 8.0, 10 mM DTT, and further denatured by adding 3 M guanidine hydrochloride, 10 mM sodium acetate, 10 mM EDTA before refolding. VVVGADGVGK peptide was added to a refolding buffer (0.4 M L-arginine, 100 mM Tris pH 8.3, 2 mM EDTA, 0.5 mM oxidized glutathione, 5 mM reduced glutathione, 0.2 mM PMSF, cooled to 4° C.) at 25 mg/L (final concentration). Then 20 mg/L of light chain and 90 mg/L of heavy chain (final concentration, heavy chain was added in three portions, 8 h/portion) were successively added, and refolded at 4° C. for at least 3 days to completion of refolding, and SDS-PAGE was used to confirm refolding.
c. Purification upon refolding: The refolding buffer was replaced with 10 volumes of 20 mM Tris pH 8.0 for dialysis, and the buffer was exchanged for at least two times to substantially reduce the ionic strength of the solution. After dialysis, the protein solution was filtered through a 0.45 μm cellulose acetate filter and loaded onto a HiTrap Q HP (GE, General Electric Company) anion exchange column (5 ml bed volume). The protein was eluted with a linear gradient of 0-400 mM NaCl prepared in 20 mM Tris pH 8.0 using Akta Purifier (GE), and the pMHC was eluted at approximately 250 mM NaCl. Peak fractions were collected and the purity thereof was detected by SDS-PAGE.
d. Biotinylation: Purified pMHC molecules were concentrated in a Millipore ultrafiltration tube, while the buffer was replaced with 20 mM Tris pH 8.0, and then biotinylation reagent 0.05 M Bicine pH 8.3, 10 mM ATP, 10 mM MgOAc, 50 μM D-Biotin, 100 μg/ml BirA enzyme (GST-BirA) was added. The resulting mixture was incubated at room temperature overnight, and SDS-PAGE was used to detect the completion of biotinylation.
e. Purification of biotinylated complex: The biotinylated and labeled pMHC molecules were concentrated to 1 ml in a Millipore ultrafiltration tube. The biotinylated pMHC was purified by gel filtration chromatography. 1 ml of concentrated biotinylated pMHC molecules was loaded on a HiPrep™ 16/60 S200 HR column (GE) pre-equilibrated with filtered PBS using an Akta Purifier (GE) and eluted with PBS at a flow rate of 1 ml/min. The biotinylated pMHC molecules were eluted as a single peak at about 55 ml. The protein-containing fractions were combined and concentrated in a Millipore ultrafiltration tube. The concentration of protein was determined by BCA method (Thermo), protease inhibitor cocktail (Roche) was added and the biotinylated pMHC molecules were dispensed and stored at −80° C.

### Example 4. Generation of High-Affinity Single-Chain TCR

Phage display technology is a means to generate high affinity TCR variant libraries for screening high affinity variants. The TCR phage display and screening method described by Li et al. ((2005) Nature Biotech 23(3): 349-354) was applied to the single-chain TCR template of Example 1. A library of high affinity TCRs was established by mutating CDR regions of the template chain and panned. After several rounds of panning, the phage library can specifically bind to the corresponding antigen, the monoclones were picked and analyzed. The CDR regions of the α chain and β chain of the high-affinity single-chain TCR obtained through screening are as follows:

| CDR | | | | | | |
|---|---|---|---|---|---|---|
| No. | α-CDR1 | α-CDR2 | α-CDR3 | β-CDR1 | β-CDR2 | β-CDR3 |
| 1 | TRDTDYY | RNSFDEQN | ALSEAGNDMR | MNHEY | SVGEGT | ASSYLWSYEQY |
| 2 | TRDTNYY | RNSFDEQN | ALSEAGNDMR | MNHEY | SVGEGT | ASSYLWSYEQY |
| 3 | TRDQNYY | RNSFDEQN | ALSEAGNDMR | MNHEY | SVGEGT | ASSYLWSYEQY |
| 4 | TRDENFY | RNSFDEQN | ALSEAGNDMR | MNHEY | SVGEGT | ASSYLWSYEQY |
| 5 | TRDENYY | RNSFDEQN | ALSEAGNDMR | MNHEY | SVGEGT | ASSYLWSYEQY |
| 6 | TRDTTYY | RNSFDEQN | ALSEAGRITN | MNHEY | SVGEGT | ASSYLWSYEQY |
| 7 | TRDTTYY | RNSFDEQN | ALSEAGRMLH | MNHEY | SVGEGT | ASSYLWSYEQY |
| 8 | TRDTTYY | RNSFDEQN | ALIEAGRTQA | MNHEY | SVGEGT | ASSYLWSYEQY |
| 9 | TRDTTYY | RNSFDEQN | ALSEAGRNTQ | MNHEY | SVGEGT | ASSYLWSYEQY |
| 10 | TRDTTYY | RNSFDEQN | ALSEAGRQLK | MNHEY | SVGEGT | ASSYLWSYEQY |
| 11 | TRDTTYY | RNSFDEQN | ALSEAGRTTH | MNHEY | SVGEGT | ASSYLWSYEQY |
| 12 | TRDTTYY | RNSFDEQN | ALSEAGRTKQ | MNHEY | SVGEGT | ASSYLWSYEQY |
| 13 | TRDTTYY | RNSFDEQN | ALSEAGRNLA | MNHEY | SVGEGT | ASSYLWSYEQY |
| 14 | TRDTTYY | RNSFDEQN | ALSEAGRNKS | MNHEY | SVGEGT | ASSYLWSYEQY |
| 15 | TRDTTYY | RNSFDEQN | ALSEAGRLTH | MNHEY | SVGEGT | ASSYLWSYEQY |
| 16 | TRDTTYY | RNSFDEQN | ALSEAGPLHS | MNHEY | SVGEGT | ASSYLWSYEQY |
| 17 | TRDTTYY | RNSFDEQN | ALSEAGRQLS | MNHEY | SVGEGT | ASSYLWSYEQY |
| 18 | TRDTTYY | RNSFDEQN | ALSEAGRMKK | MNHEY | SVGEGT | ASSYLWSYEQY |
| 19 | TRDTTYY | RNSFDEQN | ALSEAGRQKT | MNHEY | SVGEGT | ASSYLWSYEQY |
| 20 | TRDTTYY | RNSFDEQN | ALSEAGRLTA | MNHEY | SVGEGT | ASSYLWSYEQY |
| 21 | TRDTTYY | RNSFDEQN | ALSEAGRLLT | MNHEY | SVGEGT | ASSYLWSYEQY |
| 22 | TRDTTYY | RNSFDEQN | ALSEAGRQQT | MNHEY | SVGEGT | ASSYLWSYEQY |
| 23 | TRDTTYY | RNSFDEQN | ALSEAGRSKN | MNHEY | SVGEGT | ASSYLWSYEQY |
| 24 | TRDTTYY | RNSFDEQN | ALSEAGRNTD | MNHEY | SVGEGT | ASSYLWSYEQY |
| 25 | TRDTTYY | RNSFDEQN | ALSEAGRQTV | MNHEY | SVGEGT | ASSYLWSYEQY |
| 26 | TRDDNYY | RNSFDEQN | ALSEAGNDMR | MNHEY | SVGEGT | ASSYLWSYEQY |
| 27 | TRDMNYY | RNSFDEQN | ALSEAGNDMR | MNHEY | SVGEGT | ASSYLWSYEQY |
| 28 | TRDSNYY | RNSFDEQN | ALSEAGNDMR | MNHEY | SVGEGT | ASSYLWSYEQY |
| 29 | TRDVNYY | RNSFDEQN | ALSEAGNDMR | MNHEY | SVGEGT | ASSYLWSYEQY |
| 30 | TRDYNYY | RNSFDEQN | ALSEAGNDMR | MNHEY | SVGEGT | ASSYLWSYEQY |
| 31 | TRDTTYY | QTSYDEQN | ALSEAGNDMR | MNHEY | SVGEGT | ASSYLWSYEQY |
| 32 | TRDTTYY | QVSFDEQN | ALSEAGNDMR | MNHEY | SVGEGT | ASSYLWSYEQY |
| 33 | TRDTTYY | RQSWDEQN | ALSEAGNDMR | MNHEY | SVGEGT | ASSYLWSYEQY |
| 34 | TRDTTYY | RTSWDEQN | ALSEAGNDMR | MNHEY | SVGEGT | ASSYLWSYEQY |
| 35 | TRDTTYY | RNSFDEQN | ALSEAGNDMR | MNHEY | SVHEGT | ASSYLWSYEQY |
| 36 | TRDTTYY | RNSFDEQN | ALSEAGNDMR | MNHEY | SVGEET | ASSYLWSYEQY |
| 37 | TRDTTYY | RNSFDEQN | ALSEAGNDMR | MNHEY | SVGEHT | ASSYLWSYEQY |
| 38 | TRDTTYY | RNSFDEQN | ALSEAGNDMR | MNHEY | SVGEGH | ASSYLWSYEQY |
| 39 | TRDTTYY | RNSFDEQN | ALSEAGNDMR | MNHEY | SVGEDT | ASSYLWSYEQY |
| 40 | TRDTTYY | RNSFDEQN | ALSEAGNDMR | MNHEY | SVGENT | ASSYLWSYEQY |
| 41 | TRDTTYY | RNSFDEQN | ALSEAGNDMR | MNHEY | SVGEQT | ASSYLWSYEQY |
| 42 | TRDTTYY | RNSFDEQN | ALSEAGNDMR | MNHEY | SVGEKT | ASSYLWSYEQY |
| 43 | TRDTTYY | RNSFDEQN | ALSEAGNDMR | MNHEY | SVGERT | ASSYLWSYEQY |
| 44 | TRDTTYY | RNSFDEQN | ALSEAGNDMR | MNHEY | SVGQHS | ASSYLWSYEQY |
| 45 | TRDTTYY | RNSFDEQN | ALSEAGNDMR | MNHEY | SVGNTI | ASSYLWSYEQY |
| 46 | TRDTTYY | RNSFDEQN | ALSEAGNDMR | MNHEY | SVGKEI | ASSYLWSYEQY |
| 47 | TRDTTYY | RNSFDEQN | ALSEAGNDMR | MNHEY | SVGTTT | ASSYLWSYEQY |
| 48 | TRDTTYY | RNSFDEQN | ALSEAGNDMR | NNHDY | SVGEGT | ASSYLWSYEQY |

The mutation groups of the CDR regions of the screened high-affinity single-chain TCRs, i.e., CDR No. 1-48, were respectively introduced into the corresponding sites of the α and β chain variable domains of the wild-type TCR to obtain high-affinity TCRs, with the TCR No. corresponding to 1-48 respectively. Their affinities for VVVGADGVGK-HLA A1101 complex were detected by BIAcore. The mutated sites of high-affinity can be introduced in the above CDR regions by a method of site-directed mutagenesis well known to a skilled person in the art. The amino acid sequences of α chain and β chain variable domain of the above wild type TCR are shown in SEQ ID NO: 1 and SEQ ID NO: 2, respectively.

It should be noted that in order to obtain a more stable soluble TCR for easier evaluation of the binding affinity and/or binding half-life between the TCR and VVVGADGVGK-HLA A1101 complex, a cysteine residue can be introduced into the constant regions of the wild-type α and β chains, respectively to form an artificial interchain disulfide bond. In this example, the amino acid sequences of TCR α and β chains after introducing a cysteine residue are shown in SEQ ID NO: 11 and SEQ ID NO: 12, and the introduced cysteine residues are indicated by bold and double-underlined letters.

SEQ ID NO: 11
AQKVTQAQTEISVVEKEDVTLDCVYETRDTTYYLFWYKQPPSGEL
VFLIRRNSFDEQNEISGRYSWNFQKSTSSFNFTITASQVVDSAVY
FCALSEAGNDMRFGAGTRLTVKPNIQNPDPAVYQLRDSKSSDKSV
CLFTDFDSQTNVSQSKDSDVYITDKCVLDMRSMDFKSNSAVAWSN
KSDFACANAFNNSIIPEDTFFPSPESS.
SEQ ID NO: 12
NAGVTQTPKFRVLKTGQSMTLLCAQDMNHEYMYWYRQDPGMGLRL
IHYSVGEGTTAKGEVPDGYNVSRLKKQNFLLGLESAAPSQTSVYF
CASSYLWSYEQYFGPGTRLTVTEDLKNVFPPEVAVFEPSEAEISH
TQKATLVCLATGFYPDHVELSWWVNGKEVHSGVCTDPQPLKEQPA
LNDSRYALSSRLRVSATFWQDPRNHFRCQVQFYGLSENDEWTQDR
AKPVTQIVSAEAWGRAD.

According to standard methods described in Molecular Cloning a Laboratory Manual (3rd edition, Sambrook and Russell), genes of extracellular sequences of the TCR α chain and β chain to be expressed are synthesized and inserted into an expression vector pET28a+ (Novagene), in which the upstream and downstream cloning sites are NcoI and NotI, respectively. Mutations in the CDR regions are introduced by overlap PCR well known to a skilled person in the art. The inserted fragment was sequenced to confirm that it was correct.

### Example 5. Expression, Refolding and Purification of High-Affinity TCR

Expression vectors for TCR α chain and β chain were transformed into the expression bacteria BL21 (DE3) by chemical transformation, respectively. The bacteria were grown in LB medium and induced with a final concentration of 0.5 mM IPTG at OD600=0.6. The inclusion bodies formed after the TCR α and β chains were expressed were extracted by BugBuster Mix (Novagene) and repeatedly washed with BugBuster solution. The inclusion bodies were finally dissolved in a solution comprising 6 M guanidine hydrochloride, 10 mM dithiothreitol (DTT), 10 mM ethylenediaminetetraacetic acid (EDTA) and 20 mM Tris (pH 8.1).

The dissolved TCR α chain and β chain were rapidly mixed in a solution comprising 5 M urea, 0.4 M arginine, 20 mM Tris (pH 8.1), 3.7 mM cystamine, and 6.6 mM β-mercapoethylamine (4° C.) at a mass ratio of 1:1. The final concentration is 60 mg/mL. After mixing, the solution was dialyzed against 10 volumes of deionized water (4° C.), and after 12 hours, deionized water was exchanged with a buffer (20 mM Tris, pH 8.0) and dialysis was continued at 4° C. for 12 hours. After completion of the dialysis, the solution was filtered through a 0.45 μM filter and purified through an anion exchange column (HiTrap Q HP, 5 ml, GE Healthcare). The elution peak of TCR containing successfully refolded α and β dimers was confirmed by SDS-PAGE gel. The TCR was then further purified by gel filtration chromatography (HiPrep 16/60, Sephacryl S-100 HR, GE Healthcare). The purity of the purified TCR was determined by SDS-PAGE to be greater than 90%, and the concentration thereof was determined by BCA method.

### Example 6. Results of BIAcore Analysis

The affinity of the wild-type TCR, in which a high affinity CDR region was introduced, for VVVGADGVGK-HLA A1101 complex was detected by using the method described in Example 3.

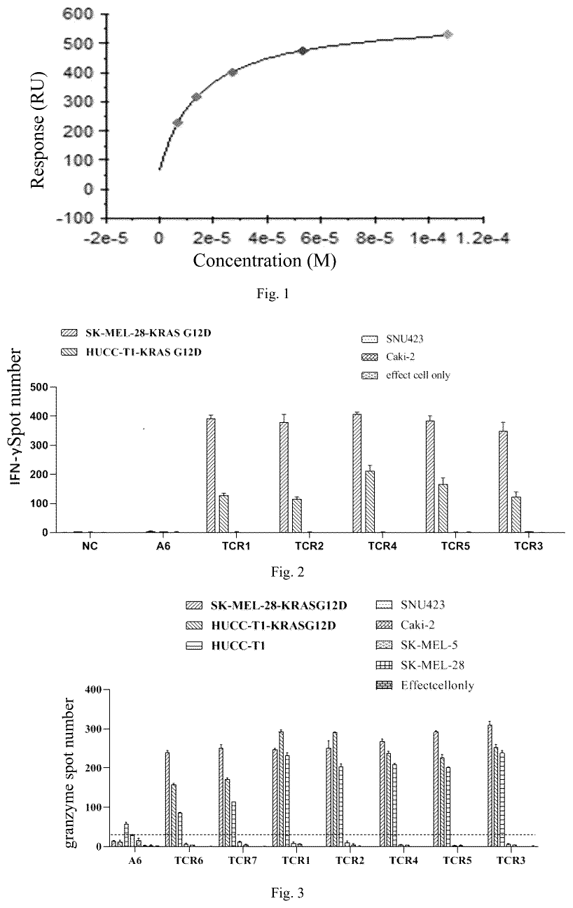

In the application, amino acid sequences of the variable domains of the newly obtained TCRα chain and TCRβ chain are shown in SEQ ID NO: 13 to SEQ ID NO: 60. Since the CDR regions of a TCR molecule determine their affinity for the corresponding pMHC complex, a skilled person in the art can anticipate that an wild-type TCR, in which a high affinity mutation site is introduced also has a high affinity for VVVGADGVGK-HLA A1101 complex. The expression vector was constructed by the method described in Example 4, the above-mentioned wild-type TCR with a high-affinity mutation being introduced was expressed, refolded and purified by the method described in Example 5, and then the affinity of the TCR for VVVGADGVGK-HLA A1101 complex is determined by BIAcore T200. The binding curve of the soluble reference TCR, i.e., the wild-type TCR, with the VVVGADGVGK-HLA A1101 complex is shown in FIG. 1. The results of affinity detection for the high-affinity TCR are shown in Table 5 below:

TABLE 5

| | α chain variable | β chain variable | |
|---|---|---|---|
| TCR No. | domain sequence | domain sequence | K_{D} |
| 1 | SEQ ID NO: 13 | SEQ ID NO: 2 | 7.78E−06 |
| 2 | SEQ ID NO: 14 | SEQ ID NO: 2 | 2.61E−06 |
| 3 | SEQ ID NO: 15 | SEQ ID NO: 2 | 1.33E−06 |
| 4 | SEQ ID NO: 16 | SEQ ID NO: 2 | 9.75E−07 |
| 5 | SEQ ID NO: 17 | SEQ ID NO: 2 | 8.00E−07 |
| 6 | SEQ ID NO: 18 | SEQ ID NO: 2 | 5.16E−06 |
| 7 | SEQ ID NO: 19 | SEQ ID NO: 2 | 4.51E−06 |
| 8 | SEQ ID NO: 20 | SEQ ID NO: 2 | 1.44E−06 |
| 9 | SEQ ID NO: 21 | SEQ ID NO: 2 | 3.42E−06 |
| 10 | SEQ ID NO: 22 | SEQ ID NO: 2 | 2.89E−06 |
| 11 | SEQ ID NO: 23 | SEQ ID NO: 2 | 2.86E−06 |
| 12 | SEQ ID NO: 24 | SEQ ID NO: 2 | 2.25E−06 |
| 13 | SEQ ID NO: 25 | SEQ ID NO: 2 | 3.70E−06 |
| 14 | SEQ ID NO: 26 | SEQ ID NO: 2 | 2.77E−06 |
| 15 | SEQ ID NO: 27 | SEQ ID NO: 2 | 3.88E−06 |
| 16 | SEQ ID NO: 28 | SEQ ID NO: 2 | 4.37E−06 |
| 17 | SEQ ID NO: 29 | SEQ ID NO: 2 | 5.24E−06 |
| 18 | SEQ ID NO: 30 | SEQ ID NO: 2 | 3.18E−06 |
| 19 | SEQ ID NO: 31 | SEQ ID NO: 2 | 4.44E−06 |
| 20 | SEQ ID NO: 32 | SEQ ID NO: 2 | 2.83E−06 |
| 21 | SEQ ID NO: 33 | SEQ ID NO: 2 | 4.55E−06 |
| 22 | SEQ ID NO: 34 | SEQ ID NO: 2 | 5.37E−06 |
| 23 | SEQ ID NO: 35 | SEQ ID NO: 2 | 4.00E−06 |
| 24 | SEQ ID NO: 36 | SEQ ID NO: 2 | 5.59E−06 |
| 25 | SEQ ID NO: 37 | SEQ ID NO: 2 | 5.35E−06 |
| 26 | SEQ ID NO: 38 | SEQ ID NO: 2 | 1.03E−06 |
| 27 | SEQ ID NO: 39 | SEQ ID NO: 2 | 1.10E−06 |
| 28 | SEQ ID NO: 40 | SEQ ID NO: 2 | 2.00E−06 |
| 29 | SEQ ID NO: 41 | SEQ ID NO: 2 | 1.65E−06 |
| 30 | SEQ ID NO: 42 | SEQ ID NO: 2 | 1.23E−06 |
| 31 | SEQ ID NO: 43 | SEQ ID NO: 2 | 2.25E−06 |
| 32 | SEQ ID NO: 44 | SEQ ID NO: 2 | 7.83E−07 |
| 33 | SEQ ID NO: 45 | SEQ ID NO: 2 | 1.26E−07 |
| 34 | SEQ ID NO: 46 | SEQ ID NO: 2 | 2.68E−07 |
| 35 | SEQ ID NO: 1 | SEQ ID NO: 47 | 8.28E−06 |
| 36 | SEQ ID NO: 1 | SEQ ID NO: 48 | 5.19E−06 |
| 37 | SEQ ID NO: 1 | SEQ ID NO: 49 | 4.65E−06 |
| 38 | SEQ ID NO: 1 | SEQ ID NO: 50 | 6.60E−06 |
| 39 | SEQ ID NO: 1 | SEQ ID NO: 51 | 8.27E−06 |
| 40 | SEQ ID NO: 1 | SEQ ID NO: 52 | 8.43E−06 |
| 41 | SEQ ID NO: 1 | SEQ ID NO: 53 | 8.18E−06 |
| 42 | SEQ ID NO: 1 | SEQ ID NO: 54 | 7.41E−06 |
| 43 | SEQ ID NO: 1 | SEQ ID NO: 55 | 7.21E−06 |
| 44 | SEQ ID NO: 1 | SEQ ID NO: 56 | 4.21E−06 |
| 45 | SEQ ID NO: 1 | SEQ ID NO: 57 | 4.33E−06 |
| 46 | SEQ ID NO: 1 | SEQ ID NO: 58 | 5.45E−06 |
| 47 | SEQ ID NO: 1 | SEQ ID NO: 59 | 3.54E−06 |
| 48 | SEQ ID NO: 1 | SEQ ID NO: 60 | 5.04E−06 |

It can be seen from the results in Table 5 that the affinities of all the high-affinity TCRs obtained in the application for the AQIPEKIQK-HLA A1101 complex have been improved.

### Example 7. Expression, Refolding and Purification of Fusions of Anti-CD3 Antibodies with High-Affinity TCR

A fusion molecule was prepared by fusing an anti-CD3 single-chain antibody (scFv) with a high-affinity TCR. The anti-CD3 scFv was fused with β chain of the TCR, and the TCR β chain may comprise β chain variable domain of any of the above high-affinity TCRs, and the TCR α chain of the fusion molecule may comprise α chain variable domain of any of the above high-affinity TCRs. The construction of the expression vector for fusion molecule includes following steps:
(1) Construction of expression vector for α chain: the target gene carrying α chain of the high-affinity TCR was digested with NcoI and NotI, and ligated with pET28a vector digested with NcoI and NotI. The ligation product was transformed into E. coli DH5α, plated on a LB plate containing kanamycin, and inverted and cultured overnight at 37° C. Positive clones were picked for PCR screening, and the positive recombinants were sequenced to determine the correct sequence. The recombinant plasmids were extracted and transformed into E. coli Tuner (DE3) for expression.
(2) Construction of expression vector for anti-CD3 (scFv)-β chain: primers were designed by overlapping PCR to connect genes of the anti-CD3 scFv and high-affinity TCRβ chain. The intermediate linker was GGGGS, and the gene fragment of the fusion protein of anti-CD3 scFv and the high-affinity TCRβ chain had the restriction endonuclease sites NcoI (CCATGG) and NotI (GCGGCCGC). The PCR amplification product was digested with NcoI and NotI and ligated with pET28a vector digested with NcoI and NotI. The ligation product was transformed into E. coli DH5a competent cells, plated on a kanamycin-containing LB plate, and inverted and cultured overnight at 37° C. Positive clones were picked for PCR screening, and the positive recombinants were sequenced to determine the correct sequence. The recombinant plasmids were extracted and transformed into E. coli Tuner (DE3) competent cells for expression.
(3) Expression, refolding and purification of fusion protein: The expression plasmids were separately transformed into E. coli Tuner (DE3) competent cells, plated on LB plates (kanamycin 50 μg/mL) and cultured overnight at 37° C. On the next day, clones were picked and inoculated into 10 mL LB liquid medium (kanamycin 50 μg/mL) for 2-3 h, and inoculated into 1 L LB medium at a volume ratio of 1:100, the culture was continued until the OD600 was 0.5-0.8, and a final concentration of 1 mM IPTG was added to induce expression of the protein of interest. After 4 hours, cells were harvested by centrifugation at 6000 rpm for 10 mins. The cells were washed once in PBS buffer and were dispensed, and cells corresponding to 200 ml of the bacterial culture were taken and lysed with 5 mL of BugBuster Master Mix (Merck), inclusion bodies were collected by centrifugation at 6000 g for 15 min and then washed with detergent for 4 times to remove cell debris and membrane components. The inclusion bodies were then washed with a buffer such as PBS to remove detergent and salt. Finally, the inclusion bodies were dissolved in 6M guanidine hydrochloride, 10 mM dithiothreitol (DTT), 10 mM ethylenediaminetetraacetic acid (EDTA), 20 mM Tris, pH 8.1 buffer solution, and the concentration of inclusion bodies was determined. The inclusion bodies were dispensed and cryopreserved at −80° C.

The dissolved TCRα chain and anti-CD3 (scFv)-β chain were rapidly mixed in a mass ratio of 2:5 in a solution comprising 5 M urea (urea), 0.4 M L-arginine (L-arginine), 20 mM Tris pH 8.1, 3.7 mM cystamine, and 6.6 mM β-mercapoethylamine (4° C.), and the final concentrations of α chain and anti-CD3 (scFv)-chain were 0.1 mg/mL, 0.25 mg/mL, respectively.

After mixing, the solution was dialyzed against 10 volumes of deionized water (4° C.), and after 12 hours, deionized water was exchanged with buffer (10 mM Tris, pH 8.0) for another 12 hours at 4° C. After completion of dialysis, the solution was filtered through a 0.45 μM filter and purified by an anion exchange column (HiTrap Q HP 5 ml, GE healthcare). The eluted peaks containing the reconstituted TCR α chain and anti-CD3 (scFv)-β chain dimer TCR were confirmed by SDS-PAGE gel. The TCR fusion molecule was then purified by size exclusion chromatography (S-100 16/60, GE healthcare) and further purified by an anion exchange column (HiTrap Q HP 5 ml, GE healthcare). The purity of the purified TCR fusion molecule was determined by SDS-PAGE to be greater than 90%, and the concentration was determined by BCA method.

### Example 8. Activation Function Experiment on the Number of IFN-γ Spots Released by Effector Cells Transfected with the High-Affinity TCR of the Application, Targeting Tumor Cell Lines

In this example, tumor cell lines were used to verify the activation function and specificity of effector cells transfected with the high-affinity TCR of the application. ELISPOT assay, which is well-known to a skilled person was also performed for detection. CD3⁺ T cells isolated from the blood of healthy volunteers, which were transfected by the high-affinity TCR of the application were used as effector cells, while CD3⁺ T cells from the same volunteer transfected with other TCRs (A6) or untransfected with TCRs (NC) were used as controls. The tumor cell lines used in the example were SK-MEL28, HUCC-T1, SNU423, and Caki-2, respectively. Among them, SK-MEL28, HUCC-T1, SNU423, and Caki-2 were all purchased from Guangzhou Saiku Biotechnology Co., Ltd. The following experiment was conducted:

The high-affinity TCRs were TCR1, TCR2, TCR3, TCR4, and TCR5, respectively. The used KRAS G12D-positive tumor cell lines were SK-MEL-28-KRAS G12D (KRAS G12D overexpression) and HUCC-T1-KRAS G12D (KRAS G12D overexpression), and the negative cell lines were SNU423, Caki-2, and those containing only effector cells.

Following steps were performed: Firstly, a ELISPOT plate was prepared. The ELISPOT plate was activated with ethanol and coated overnight at 4° C. On the first day of the experiment, the coating solution was removed, and the plate was washed, blocked and incubated at room temperature for 2 hrs, and the blocking solution was removed. Components of the assay were added to the ELISPOT plate: target cells 2×10⁴ cells/ml, effector cells 10³ cells/ml (Calculated according to the positive rate of transfection) in duplicate, and incubate overnight (37° C., 5% CO2). On the second day of the experiment, the plate was washed, subjected to a secondary detection and development, and dried, and the spots formed on the film were counted using an immunospot plate reader (ELISPOT READER system; AID20 company).

FIG. 2 shows the results of the activation function experiment of effector cells transfected with the high-affinity TCR of the application targeting tumor cell lines. The results shown in FIG. 2 indicate that, for KRAS G12D-positive tumor cell lines, the effector cells transfected with the high-affinity TCR of the application exhibited very obvious activation effects, while the effector cells transfected with other TCRs (A6) or without TCR transfection (NC) showed basically no activity; meanwhile, the effector cells transfected with the high-affinity TCR of the application showed basically no activity against KRAS G12D-negative cell lines.

### Example 9. Activation Function Experiment on the Release of Granzyme Spots from Effector Cells Transfected with the High-Affinity TCR of the Application, Targeting Tumor Cell Lines

In this example, tumor cell lines were used again to verify the activation function and specificity of effector cells transfected with the high-affinity TCR of the application. ELISPOT assay, which is well-known to a skilled person was also performed for detection. CD3⁺ T cells isolated from the blood of healthy volunteers, which were transfected by the high-affinity TCR of the application were used as effector cells, while CD3⁺ T cells from the same volunteer transfected with other TCRs (A6) or untransfected with TCRs (NC) were used as controls. The tumor cell lines used in the example were SK-MEL28, HUCC-T1, SNU423, Caki-2 and SK-MEL-5, respectively. Among them, SK-MEL28, HUCC-T1, SNU423, Caki-2 and SK-MEL-5 were all purchased from Guangzhou Saiku Biotechnology Co., Ltd, and SK-MEL-5 was purchased from ATCC. The following experiment was conducted:

The high-affinity TCRs were TCR1, TCR2, TCR3, TCR4, TCR5, TCR6 and TCR7, respectively. The used KRAS G12D-positive tumor cell lines were SK-MEL-28-KRAS G12D (KRAS G12D overexpression) and HUCC-T1-KRAS G12D (KRAS G12D overexpression), and HUCC-T1, and the negative cell lines were SNU423, Caki-2, SK-MEL-5, SK-MEL-28 and those containing only effector cells.

Following steps were performed: Firstly, a ELISPOT plate was prepared. The ELISPOT plate was activated with ethanol and coated overnight at 4° C. On the first day of the experiment, the coating solution was removed, and the plate was washed, blocked and incubated at room temperature for 2 hrs, and the blocking solution was removed. Components of the assay were added to the ELISPOT plate: target cells 2×10⁴ cells/ml, effector cells 10³ cells/ml (Calculated according to the positive rate of transfection) in duplicate, and incubate overnight (37° C., 5% CO2). On the second day of the experiment, the plate was washed, subjected to a secondary detection and development, and dried, and the spots formed on the film were counted using an immunospot plate reader (ELISPOT READER system; AID20 company).

FIG. 3 shows the results of the activation function experiment of effector cells transfected with the high-affinity TCR of the application targeting tumor cell lines. The results shown in FIG. 3 indicate that, for KRAS G12D-positive tumor cell lines, the effector cells transfected with the high-affinity TCR of the application exhibited very obvious activation effects, while the effector cells transfected with other TCRs (A6) showed basically no activity; meanwhile, the effector cells transfected with the high-affinity TCR of the application showed basically no activity against KRAS G12D-negative cell lines.

### Example 10. Experiment on the Killing Function of Effector Cells Transfected with the High-Affinity TCR of the Application Against Tumor Cell Lines

Lactate dehydrogenase (LDH) is abundant in the cytoplasm and cannot pass through the cell membrane under normal circumstances. When cells are damaged or die, it can be released outside the cells. At this time, the LDH activity in the cell culture medium is proportional to the number of dead cells. In this example, the release of LDH was also determined through a non-radioactive cytotoxicity assay well-known to a skilled person, so as to verify the killing function of the cells transfected with the TCR of the application. In the LDH experiment of this example, CD3⁺ T cells isolated from the blood of healthy volunteers transfected with the high-affinity TCR of the application were used as effector cells, and CD3⁺ T cells transfected with other TCRs (A6) from the same volunteer were used as controls. The tumor cell lines used in the examples were SK-MEL28 and Caki-2, respectively. Among them, SK-MEL28 and Caki-2 were purchased from Guangzhou Saiku Biotechnology Co., Ltd. The following experiment was conducted:

The high-affinity TCRs were TCR1, TCR2, TCR3, TCR4, TCR5, TCR6, and TCR7, respectively. The KRAS G12D-positive tumor cell line used was SK-MEL-28-KRAS G12D (with overexpression of KRAS G12D), and the negative tumor cell lines were SK-MEL28 and Caki-2.

Experimental steps: First, a LDH plate was prepared. Each component of the experiment was added to the plate in the following order: target cells at 3×10⁴ cells/well, effector cells at 3×10⁴ cells/well (calculated based on transfection positive rate), were added to the corresponding wells in triplicate. Meanwhile, effector cell spontaneous release wells, target cell spontaneous release wells, target cell maximum release wells, volume correction control wells, and medium background control wells were set up. The plate was incubated overnight (37° C., 5% CO2). On the second day of the experiment, the color development was detected. After the reaction was quenched, the absorbance at 490 nm was recorded using a microplate reader (Bioteck).

FIG. 4 shows the results of the LDH assay for killing function of effector cells transfected with the high-affinity TCR of the application targeting tumor cell lines. The results shown in FIG. 4 indicate that, for KRAS G12D-positive tumor cell lines, the effector cells transfected with the high-affinity TCR of the application showed significantly strong killing efficacy, while the T cells transfected with other TCRs (A6) basically exhibited no response. Meanwhile, the T cells transfected with the high-affinity TCR of the application showed almost no killing effects on negative tumor cell lines.

### Example 11. ELISA Activation Function Experiment of Effector Cells Transfected with High-Affinity TCR of the Application Targeting Tumor Cell Lines

To verify the activation function and specificity of the effector cells transfected with the high-affinity TCR of the application again, in this example, tumor cell lines were used to conduct ELISA experiments. CD3⁺ T cells isolated from the blood of healthy volunteers transfected by the high-affinity TCR of the application were used as effector cells, and CD3⁺ T cells transfected with other TCRs (A6) from the same volunteer were used as negative controls. The tumor cell lines used in the example were SK-MEL-28 and Caki-2. Among them, SK-MEL-28 and Caki-2 were purchased from Guangzhou Saiku Biotechnology Co., Ltd. The following experiment was conducted:

The high-affinity TCRs were TCR1, TCR2, TCR3, TCR4, TCR5, TCR6, and TCR7, respectively. The used KRAS G12D-positive tumor cell line was SK-MEL-28-KRAS G12D (with KRAS G12D overexpression), and the negative tumor cell lines were SK-MEL28, Caki-2, as well as those containing only effector cells.

Experimental steps: On the first day of the experiment, the cells were seeded: the tumor cell line and the cell suspension of the TCR in the application were inoculated into a U-shaped plate: the target cells were 3×10⁴ cells/well, and the effector cells were 9×10⁴ cells/well (calculated according to the positive rate of transfection), in triplicate, and incubated overnight (37° C., 5% CO₂). The ELISA plate was coated with IL-2 using antibodies, and placed in a 4° C. refrigerator overnight. On the second day of the experiment, the coating solution was removed, the plate was washed, blocked and incubated at room temperature for two hours, and then the blocking solution was removed. The co-supernatant of the tumor cell line and the TCR of the application was added to the ELISA plate, and a standard protein was taken, diluted at a 10-fold ratio, added to the ELISA plate, and placed on a shaker at room temperature for two hours. The plate was washed, biotin-labeled secondary antibody was added, and placed on a shaker at room temperature for one hour and washed. SA-HRP was added, placed on a shaker at room temperature for one hour, and washed. TMB was added for color development for 5-10 minutes, the reaction was quenched, and detected at a wavelength of 450 nm.

FIG. 5 shows the results of an ELISA experiment on the killing function of effector cells transfected with the high-affinity TCR of the application against tumor cell lines. The results in FIG. 5 indicate that, for KRAS G12D-positive tumor cell lines, the effector cells transfected with the high-affinity TCR of the application exhibited a significant activation response, while T cells transfected with other TCRs (A6) showed almost no response. Additionally, T cells transfected with the high-affinity TCR of the application exhibited almost no killing effects on negative tumor cell lines.

### Example 12. Experiment on the Killing Function of Effector Cells Transfected with the High-Affinity TCR of the Application Against Tumor Cell Lines (IncuCyte Experiment)

In this example, the specific killing effect and sensitivity of effector cells transfected with the high-affinity TCR of the application on target cells were further verified through the IncuCyte experiment well-known to a skilled person. IncuCyte is a functional analysis system that can automatically analyze images at different time points, and quantify the number of real-time cell apoptosis by performing real-time microscopic imaging in an incubator.

CD3⁺ T cells isolated from the blood of healthy volunteers transfected with the TCR of the application were randomly selected as effector cells, and the experimental groups transfected with other TCRs (A6) from the same volunteer or with only target cells (Target cell only) were used as control groups. The high-affinity TCRs were TCR1, TCR2, TCR3, TCR4, TCR5, TCR6, and TCR7 respectively. The used positive tumor cell line was SK-MEL-28-KRAS G12D (with overexpression of KRAS G12D); and the negative tumor cell line was SNU423.

On the first day of the experiment, the target cells were digested and centrifuged; resuspended in complete medium consisting of phenol red-free RPMI1640+10% FBS, and evenly plated in a 96-well plate at a density of 2×10⁴ cells per well. The plate was then placed back into a 37° C., 5% CO₂ incubator and incubated overnight. On the second day, the medium in the 96-well plate was discarded and replaced with phenol red-free RPMI1640+10% FBS medium containing caspase3/7 reagent dye, with the dye concentration adjusted to 2 drops per ml. The old medium was discarded, and new phenol red-free RPMI1640+10% FBS medium was added. Effector cells were co-incubated with the experimental group with target cells being plated at a density of 1×10⁴ cells per well (calculated based on transfection positive rate). The plate was placed into IncuCyte ZooM, a real-time dynamic live cell imaging analyzer dedicated to Incucyte detection, and after 30 minutes of incubation, real-time observation and photography were started. IncuCyte ZooM 2016A was used to process, analyze, and export the detection results.

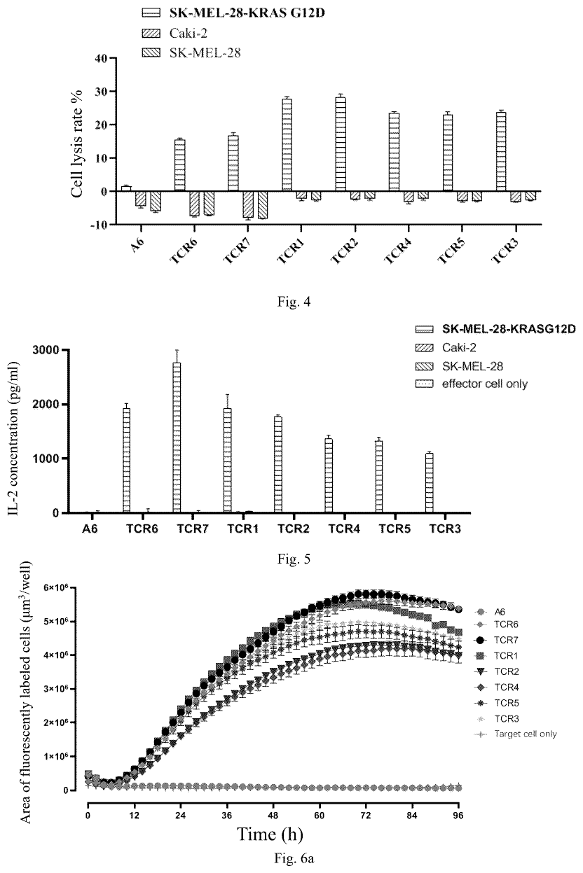

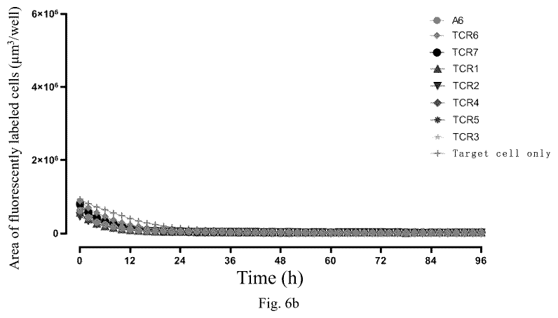

FIGS. 6a and 6b show the results of the IncuCyte experiment on the killing function of effector cells transfected with the high-affinity TCR of the application against tumor cell lines. The results in FIGS. 6a and 6b indicate that, for KRAS G12D-positive tumor cell lines, the effector cells transfected with the high-affinity TCR of the application can exhibit significantly strong killing efficacy in a short period of time, while T cells transfected with other TCRs basically exhibited no response. Meanwhile, the T cells transfected with the high-affinity TCR of the application have almost no killing effect on negative tumor cell lines.

All documents mentioned in the application are cited as references in this application, just as each document is individually cited as a reference. In addition, it should be understood that after reading the above teaching contents of the application, a skilled person can make various changes or modifications to the application, and these equivalent forms also fall within the scope defined by the appended claims of the application.

The applicant states that the above descriptions are only specific embodiment of the application, but the protection scope of the application is not limited thereto. A skilled person in the relevant technical field should understand that any changes or substitutions that can be easily conceived by a skilled person within the technical scope disclosed in the application shall fall within the protection scope and disclosed scope of the application.

1. A T cell receptor (TCR) comprising a TCRα chain variable domain and a TCRβ chain variable domain, wherein the T cell receptor has the activity of binding to the VVVGADGVGK-HLA A1101 complex;
and the amino acid sequence of the TCRα chain variable domain has at least 90% sequence homology with the amino acid sequence shown in SEQ ID NO: 1, and the amino acid sequence of the TCRβ chain variable domain has at least 90% sequence homology with the amino acid sequence shown in SEQ ID NO: 2.

2. The TCR of claim 1, wherein, in the TCRβ chain variable domain, the CDR1β is MNHEY, the CDR2β is SVGEGT, and the CDR3β is ASSYLWSYEQY.

3. The TCR of claim 1, wherein the amino acid sequence of the TCRβ chain variable domain is SEQ ID NO: 2.

4. The TCR of claim 1, wherein the amino acid sequence of the TCRα chain variable domain has at least 95% sequence homology with the amino acid sequence shown in SEQ ID NO: 1, and the amino acid sequence of the TCRβ chain variable domain has at least 95% sequence homology with the amino acid sequence shown in SEQ ID NO: 2.

5. The TCR of claim 1, wherein the reference sequences of the three CDRs (complementarity determining regions) in the TCRα chain variable domain are as follows:
CDR1α:
SEQ ID NO: 65
TRDTTYY
CDR2α:
SEQ ID NO: 66
RNSFDEQN
CDR3α:
SEQ ID NO: 67
ALSEAGNDMR,
and also comprises at least one mutation from following:

| Residue before mutation | Residue after mutation |
|---|---|
| T at position 4 | E or Q or D or M or S or V or Y |
| of CDR1α | |
| T at position 5 | N or D |
| of CDR1α | |
| Y at position 6 | F |
| of CDR1α | |
| R at position 1 | Q |
| of CDR2α | |
| N at position 2 | T or V or Q |
| of CDR2α | |
| F at position 4 | Y or W |
| of CDR2α | |
| N at position 7 | R |
| of CDR3α | |
| D at position 8 | I or M or T or N or Q or L or S |
| of CDR3α | |
| M at position 9 | T or L or Q or K or H |
| of CDR3α | |
| R at position 10 | N or H or A or Q or K or S or T or D or V. |
| of CDR3α | |

6. The TCR of claim 1, wherein the reference sequences of the three CDRs (complementarity determining regions) in the TCRβ chain variable domain are as follows:
CDR1β: MNHEY
CDR2β: SVGEGT
CDR3β: ASSYLWSYEQY
and comprises at least one mutation from following:

| Residue before mutation | Residue after mutation |
|---|---|
| M at position 1 | N |
| of CDR1β | |
| E at position 4 | D |
| of CDR1β | |
| G at position 3 | H |
| of CDR2β | |
| E at position 4 | Q or N or K or T |
| of CDR2β | |
| G at position 5 | E or H or D or N or Q or K or R or T |
| of CDR2β | |
| T at position 6 | H or S or I. |
| of CDR2β | |

7. The TCR of claim 1, wherein the TCR has CDRs selected from the following group:

| CDR | | | | | | |
|---|---|---|---|---|---|---|
| number | α-CDR1 | α-CDR2 | α-CDR3 | β-CDR1 | β-CDR2 | β-CDR3 |
| 1 | TRDTDYY | RNSFDEQN | ALSEAGNDMR | MNHEY | SVGEGT | ASSYLWSYEQY |
| 2 | TRDTNYY | RNSFDEQN | ALSEAGNDMR | MNHEY | SVGEGT | ASSYLWSYEQY |
| 3 | TRDQNYY | RNSFDEQN | ALSEAGNDMR | MNHEY | SVGEGT | ASSYLWSYEQY |
| 4 | TRDENFY | RNSFDEQN | ALSEAGNDMR | MNHEY | SVGEGT | ASSYLWSYEQY |
| 5 | TRDENYY | RNSFDEQN | ALSEAGNDMR | MNHEY | SVGEGT | ASSYLWSYEQY |
| 6 | TRDTTYY | RNSFDEQN | ALSEAGRITN | MNHEY | SVGEGT | ASSYLWSYEQY |
| 7 | TRDTTYY | RNSFDEQN | ALSEAGRMLH | MNHEY | SVGEGT | ASSYLWSYEQY |
| 8 | TRDTTYY | RNSFDEQN | ALIEAGRTQA | MNHEY | SVGEGT | ASSYLWSYEQY |
| 9 | TRDTTYY | RNSFDEQN | ALSEAGRNTQ | MNHEY | SVGEGT | ASSYLWSYEQY |
| 10 | TRDTTYY | RNSFDEQN | ALSEAGRQLK | MNHEY | SVGEGT | ASSYLWSYEQY |
| 11 | TRDTTYY | RNSFDEQN | ALSEAGRTTH | MNHEY | SVGEGT | ASSYLWSYEQY |
| 12 | TRDTTYY | RNSFDEQN | ALSEAGRTKQ | MNHEY | SVGEGT | ASSYLWSYEQY |
| 13 | TRDTTYY | RNSFDEQN | ALSEAGRNLA | MNHEY | SVGEGT | ASSYLWSYEQY |
| 14 | TRDTTYY | RNSFDEQN | ALSEAGRNKS | MNHEY | SVGEGT | ASSYLWSYEQY |
| 15 | TRDTTYY | RNSFDEQN | ALSEAGRLTH | MNHEY | SVGEGT | ASSYLWSYEQY |
| 16 | TRDTTYY | RNSFDEQN | ALSEAGPLHS | MNHEY | SVGEGT | ASSYLWSYEQY |
| 17 | TRDTTYY | RNSFDEQN | ALSEAGRQLS | MNHEY | SVGEGT | ASSYLWSYEQY |
| 18 | TRDTTYY | RNSFDEQN | ALSEAGRMKK | MNHEY | SVGEGT | ASSYLWSYEQY |
| 19 | TRDTTYY | RNSFDEQN | ALSEAGRQKT | MNHEY | SVGEGT | ASSYLWSYEQY |
| 20 | TRDTTYY | RNSFDEQN | ALSEAGRLTA | MNHEY | SVGEGT | ASSYLWSYEQY |
| 21 | TRDTTYY | RNSFDEQN | ALSEAGRLLT | MNHEY | SVGEGT | ASSYLWSYEQY |
| 22 | TRDTTYY | RNSFDEQN | ALSEAGRQQT | MNHEY | SVGEGT | ASSYLWSYEQY |
| 23 | TRDTTYY | RNSFDEQN | ALSEAGRSKN | MNHEY | SVGEGT | ASSYLWSYEQY |
| 24 | TRDTTYY | RNSFDEQN | ALSEAGRNTD | MNHEY | SVGEGT | ASSYLWSYEQY |
| 25 | TRDTTYY | RNSFDEQN | ALSEAGRQTV | MNHEY | SVGEGT | ASSYLWSYEQY |
| 26 | TRDDNYY | RNSFDEQN | ALSEAGNDMR | MNHEY | SVGEGT | ASSYLWSYEQY |
| 27 | TRDMNYY | RNSFDEQN | ALSEAGNDMR | MNHEY | SVGEGT | ASSYLWSYEQY |
| 28 | TRDSNYY | RNSFDEQN | ALSEAGNDMR | MNHEY | SVGEGT | ASSYLWSYEQY |
| 29 | TRDVNYY | RNSFDEQN | ALSEAGNDMR | MNHEY | SVGEGT | ASSYLWSYEQY |
| 30 | TRDYNYY | RNSFDEQN | ALSEAGNDMR | MNHEY | SVGEGT | ASSYLWSYEQY |
| 31 | TRDTTYY | QTSYDEQN | ALSEAGNDMR | MNHEY | SVGEGT | ASSYLWSYEQY |
| 32 | TRDTTYY | QVSFDEQN | ALSEAGNDMR | MNHEY | SVGEGT | ASSYLWSYEQY |
| 33 | TRDTTYY | RQSWDEQN | ALSEAGNDMR | MNHEY | SVGEGT | ASSYLWSYEQY |
| 34 | TRDTTYY | RTSWDEQN | ALSEAGNDMR | MNHEY | SVGEGT | ASSYLWSYEQY |
| 35 | TRDTTYY | RNSFDEQN | ALSEAGNDMR | MNHEY | SVHEGT | ASSYLWSYEQY |
| 36 | TRDTTYY | RNSFDEQN | ALSEAGNDMR | MNHEY | SVGEET | ASSYLWSYEQY |
| 37 | TRDTTYY | RNSFDEQN | ALSEAGNDMR | MNHEY | SVGEHT | ASSYLWSYEQY |
| 38 | TRDTTYY | RNSFDEQN | ALSEAGNDMR | MNHEY | SVGEGH | ASSYLWSYEQY |
| 39 | TRDTTYY | RNSFDEQN | ALSEAGNDMR | MNHEY | SVGEDT | ASSYLWSYEQY |
| 40 | TRDTTYY | RNSFDEQN | ALSEAGNDMR | MNHEY | SVGENT | ASSYLWSYEQY |
| 41 | TRDTTYY | RNSFDEQN | ALSEAGNDMR | MNHEY | SVGEQT | ASSYLWSYEQY |
| 42 | TRDTTYY | RNSFDEQN | ALSEAGNDMR | MNHEY | SVGEKT | ASSYLWSYEQY |
| 43 | TRDTTYY | RNSFDEQN | ALSEAGNDMR | MNHEY | SVGERT | ASSYLWSYEQY |
| 44 | TRDTTYY | RNSFDEQN | ALSEAGNDMR | MNHEY | SVGQHS | ASSYLWSYEQY |
| 45 | TRDTTYY | RNSFDEQN | ALSEAGNDMR | MNHEY | SVGNTI | ASSYLWSYEQY |
| 46 | TRDTTYY | RNSFDEQN | ALSEAGNDMR | MNHEY | SVGKEI | ASSYLWSYEQY |
| 47 | TRDTTYY | RNSFDEQN | ALSEAGNDMR | MNHEY | SVGTTT | ASSYLWSYEQY |
| 48 | TRDTTYY | RNSFDEQN | ALSEAGNDMR | NNHDY | SVGEGT | ASSYLWSYEQY. |

8. The TCR of claim 1, wherein the TCR is soluble.

9. The TCR of claim 1, wherein the TCR is an αβ heterodimeric TCR, which comprises the α chain TRAC constant region sequence and the β chain TRBC1 or TRBC2 constant region sequence.

10. The TCR of claim 1, wherein the TCR comprises (i) TCR α chain variable domain and all or part of the TCR α chain constant region except for transmembrane domain, and (ii) TCR β chain variable domain and all or part of the TCR β chain constant region except for transmembrane domain.

11. The TCR of claim 1, wherein the TCR comprises α chain constant region and β chain constant region, and an artificial interchain disulfide bond is contained between the α chain constant region and the β chain constant region of the TCR;
and optionally, cysteine residues forming an artificial interchain disulfide bond between the α chain constant region and the β chain constant region of the TCR are substituted for one or more groups of amino acids selected from the following:
Thr48 in exon 1 of TRAC*01 and Ser57 in exon 1 of TRBC1*01 or TRBC2*01;
Thr45 in exon 1 of TRAC*01 and Ser77 in exon 1 of TRBC1*01 or TRBC2*01;
Tyr10 in exon 1 of TRAC*01 and Ser17 in exon 1 of TRBC1*01 or TRBC2*01;
Thr45 in exon 1 of TRAC*01 and Asp59 in exon 1 of TRBC1*01 or TRBC2*01;
Ser15 in exon 1 of TRAC*01 and Glu15 in exon 1 of TRBC1*01 or TRBC2*01;
Arg53 in exon 1 of TRAC*01 and Ser54 in exon 1 of TRBC1*01 or TRBC2*01;
Pro89 in exon 1 of TRAC*01 and Ala19 in exon 1 of TRBC1*01 or TRBC2*01;
and Tyr10 in exon 1 of TRAC*01 and Glu20 in exon 1 of TRBC1*01 or TRBC2*01.

12. The TCR of claim 1, wherein the TCR satisfies one or more of the conditions shown in (I) and (II) below:
(I) the amino acid sequence of the TCR α chain variable domain is selected from SEQ ID NO: 1, SEQ ID NO: 13-46; and
(II) the amino acid sequence of the TCR β chain variable domain is selected from SEQ ID NO: 2, SEQ ID NO: 47-60;
Optionally, the TCR is selected from the following group:
(1) the sequence of the α chain variable domain is SEQ ID NO:13, and the sequence of the β chain variable domain is SEQ ID NO:2
(2) the sequence of the α chain variable domain is SEQ ID NO:14, and the sequence of the β chain variable domain is SEQ ID NO:2
(3) the sequence of the α chain variable domain is SEQ ID NO:15, and the sequence of the β chain variable domain is SEQ ID NO:2
(4) the sequence of the α chain variable domain is SEQ ID NO:16, and the sequence of the β chain variable domain is SEQ ID NO:2
(5) the sequence of the α chain variable domain is SEQ ID NO:17, and the sequence of the β chain variable domain is SEQ ID NO:2
(6) the sequence of the α chain variable domain is SEQ ID NO:18, and the sequence of the β chain variable domain is SEQ ID NO: 2
(7) the sequence of the α chain variable domain is SEQ ID NO: 19, and the sequence of the β chain variable domain is SEQ ID NO:2
(8) the sequence of the α chain variable domain is SEQ ID NO:20, and the sequence of the β chain variable domain is SEQ ID NO:2
(9) the sequence of the α chain variable domain is SEQ ID NO:21, and the sequence of the β chain variable domain is SEQ ID NO:2
(10) the sequence of the α chain variable domain is SEQ ID NO:22, and the sequence of the β chain variable domain is SEQ ID NO:2
(11) the sequence of the α chain variable domain is SEQ ID NO:23, and the sequence of the β chain variable domain is SEQ ID NO:2
(12) the sequence of the α chain variable domain is SEQ ID NO:24, and the sequence of the β chain variable domain is SEQ ID NO:2
(13) the sequence of the α chain variable domain is SEQ ID NO:25, and the sequence of the β chain variable domain is SEQ ID NO:2
(14) the sequence of the α chain variable domain is SEQ ID NO:26, and the sequence of the β chain variable domain is SEQ ID NO:2
(15) the sequence of the α chain variable domain is SEQ ID NO:27, and the sequence of the β chain variable domain is SEQ ID NO:2
(16) the sequence of the α chain variable domain is SEQ ID NO:28, and the sequence of the β chain variable domain is SEQ ID NO:2
(17) the sequence of the α chain variable domain is SEQ ID NO:29, and the sequence of the β chain variable domain is SEQ ID NO:2
(18) the sequence of the α chain variable domain is SEQ ID NO:30, and the sequence of the β chain variable domain is SEQ ID NO:2
(19) the sequence of the α chain variable domain is SEQ ID NO:31, and the sequence of the β chain variable domain is SEQ ID NO:2
(20) the sequence of the α chain variable domain is SEQ ID NO:32, and the sequence of the β chain variable domain is SEQ ID NO:2
(21) the sequence of the α chain variable domain is SEQ ID NO:33, and the sequence of the β chain variable domain is SEQ ID NO:2
(22) the sequence of the α chain variable domain is SEQ ID NO:34, and the sequence of the β chain variable domain is SEQ ID NO:2
(23) the sequence of the α chain variable domain is SEQ ID NO:35, and the sequence of the β chain variable domain is SEQ ID NO:2
(24) the sequence of the α chain variable domain is SEQ ID NO:36, and the sequence of the β chain variable domain is SEQ ID NO:2
(25) the sequence of the α chain variable domain is SEQ ID NO:37, and the sequence of the β chain variable domain is SEQ ID NO:2
(26) the sequence of the α chain variable domain is SEQ ID NO:38, and the sequence of the β chain variable domain is SEQ ID NO:2
(27) the sequence of the α chain variable domain is SEQ ID NO:39, and the sequence of the β chain variable domain is SEQ ID NO:2
(28) the sequence of the α chain variable domain is SEQ ID NO:40, and the sequence of the β chain variable domain is SEQ ID NO:2
(29) the sequence of the α chain variable domain is SEQ ID NO:41, and the sequence of the β chain variable domain is SEQ ID NO:2
(30) the sequence of the α chain variable domain is SEQ ID NO:42, and the sequence of the β chain variable domain is SEQ ID NO:2
(31) the sequence of the α chain variable domain is SEQ ID NO:43, and the sequence of the β chain variable domain is SEQ ID NO:2
(32) the sequence of the α chain variable domain is SEQ ID NO:44, and the sequence of the β chain variable domain is SEQ ID NO:2
(33) the sequence of the α chain variable domain is SEQ ID NO:45, and the sequence of the β chain variable domain is SEQ ID NO:2
(34) the sequence of the α chain variable domain is SEQ ID NO:46, and the sequence of the β chain variable domain is SEQ ID NO:2
(35) the sequence of the α chain variable domain is SEQ ID NO:1, and the sequence of the β chain variable domain is SEQ ID NO:47
(36) the sequence of the α chain variable domain is SEQ ID NO:1, and the sequence of the β chain variable domain is SEQ ID NO:48
(37) the sequence of the α chain variable domain is SEQ ID NO:1, and the sequence of the β chain variable domain is SEQ ID NO:49
(38) the sequence of the α chain variable domain is SEQ ID NO:1, and the sequence of the β chain variable domain is SEQ ID NO:50
(39) the sequence of the α chain variable domain is SEQ ID NO:1, and the sequence of the β chain variable domain is SEQ ID NO:51
(40) the sequence of the α chain variable domain is SEQ ID NO:1, and the sequence of the β chain variable domain is SEQ ID NO:52
(41) the sequence of the α chain variable domain is SEQ ID NO:1, and the sequence of the β chain variable domain is SEQ ID NO:53
(42) the sequence of the α chain variable domain is SEQ ID NO:1, and the sequence of the β chain variable domain is SEQ ID NO:54
(43) the sequence of the α chain variable domain is SEQ ID NO:1, and the sequence of the β chain variable domain is SEQ ID NO:55
(44) the sequence of the α chain variable domain is SEQ ID NO:1, and the sequence of the β chain variable domain is SEQ ID NO:56
(45) the sequence of the α chain variable domain is SEQ ID NO:1, and the sequence of the β chain variable domain is SEQ ID NO:57
(46) the sequence of the α chain variable domain is SEQ ID NO:1, and the sequence of the β chain variable domain is SEQ ID NO:58
(47) the sequence of the α chain variable domain is SEQ ID NO:1, and the sequence of the β chain variable domain is SEQ ID NO:59, and
(48) the sequence of the α chain variable domain is SEQ ID NO:1, and the sequence of the β chain variable domain is SEQ ID NO:60.

13. The TCR of claim 1, wherein the TCR is a single chain TCR;
optionally, the TCR is a single-chain TCR consisting of an α chain variable domain and a β chain variable domain, and the α chain variable domain and the β chain variable domain are connected by a flexible short peptide sequence (linker).

14. The TCR of claim 1, wherein the TCR satisfies one or more of the conditions shown in (I) and (II) below:
(I) a conjugate binds to the α chain of the TCR at C- or N-terminal; and
(II) a conjugate binds to the β chain of the TCR at C- or N-terminal;
optionally, the conjugate is a detectable label or a therapeutic agent; and further optionally, the therapeutic agent is an anti-CD3 antibody.

15. A multivalent TCR complex comprising at least two TCR molecules, and at least one TCR molecule is the TCR of claim 1.

16. A nucleic acid molecule, comprising a nucleic acid sequence encoding the TCR of claim 1, or a complement sequence thereof.

17. A vector, comprising the nucleic acid molecule of claim 16.

18. A host cell, comprising the vector of claim 17 or having the exogenous nucleic acid molecule of a nucleic acid molecule, comprising a nucleic acid sequence encoding the TCR of a T cell receptor (TCR) comprising a TCRα chain variable domain and a TCRβ chain variable domain integrated into its genome.

19. An isolated cell, expressing the TCR of claim 1, and optionally, the isolated cell is a T cell.

20. An isolated cell, wherein the isolated cell expresses the TCR of claim 1 and also expressing an exogenous CD8 receptor; optionally, the CD8 receptor is CD8α; and further optionally, the isolated cell is a T cell.

21. A pharmaceutical composition, comprising the TCR of claim 1, or the TCR complex of a multivalent TCR complex comprising at least two TCR molecules, or the isolated cell of an isolated cell, expressing the TCR of a T cell receptor (TCR) comprising a TCRα chain variable domain and a TCRβ chain variable domain.

22. A method for treating a disease, comprising administering the TCR of claim 1, or the TCR complex of a multivalent TCR complex comprising at least two TCR molecules, or the cell of an isolated cell, expressing the TCR of a T cell receptor (TCR) comprising a TCRα chain variable domain and a TCRβ chain variable domain, or the pharmaceutical composition of A pharmaceutical composition, comprising the TCR of a T cell receptor (TCR) comprising a TCRα chain variable domain and a TCRβ chain variable domain to a subject in need thereof; optionally, the disease is a KRAS G12D-positive tumor, and further optionally, the disease is colorectal cancer, pancreatic cancer, or gastric cancer.

23. Use of the TCR of claim 1, or the TCR complex of a multivalent TCR complex at least two TCR molecules, or the isolated cell of an isolated cell expressing the TCR of a T cell receptor (TCR) comprising a TCRα chain variable domain and a TCRβ chain variable domain for preparing a specific binding reagent for a target protein.

24. The use of claim 23, wherein the specific binding reagent for a target protein is a diagnostic agent and therapeutic agent for a tumor expressing the target protein; and optionally, the therapeutic agent is directed against a KRAS G12D-positive tumor.

25. Use of the TCR of claim 1, or the multivalent TCR complex of a multivalent TCR complex comprising at least two TCR molecules, or the isolated cell of an isolated cell, expressing the TCR of a T cell receptor (TCR) comprising a TCRα chain variable domain and a TCRβ chain variable domain for preparing a medicament for treating a tumor; optionally, the tumor is a KRAS G12D-positive tumor; and further optionally, the tumor is colorectal cancer, pancreatic cancer, or gastric cancer.

26. A method for preparing the T cell receptor of claim 1, comprising the steps of:
(i) culturing the cell of a host cell, comprising the vector of vector, comprising the nucleic acid molecule to express the T cell receptor of claim 1;
(ii) isolating or purifying the T cell receptor.
